(19) United States
(12) Patent Application Publication
(10) Pub. No.: US 2020/0046648 A1
(43) Pub. Date: Feb. 13, 2020

(54) POLYMERIC NANOPARTICLES COMPRISING SALINOMYCIN

(71) Applicants: SURENDER KHARBANDA, BOCA RATON, FL (US); JAMES HILL, BOCA RATON, FL (US); SIREESH APPAJOSYULAN, BOCA RATON, FL (US); MARK ROSENBERG, BOCA RATON, FL (US); HARPAL SINGH, BOCA RATON, FL (US)

(72) Inventors: SURENDER KHARBANDA, BOCA RATON, FL (US); JAMES HILL, BOCA RATON, FL (US); SIREESH APPAJOSYULAN, BOCA RATON, FL (US); MARK ROSENBERG, BOCA RATON, FL (US); HARPAL SINGH, BOCA RATON, FL (US)

(21) Appl. No.: 16/515,405

(22) Filed: Jul. 18, 2019

Related U.S. Application Data
(60) Provisional application No. 62/699,963, filed on Jul. 18, 2018.

(51) Int. Cl. A61K 9/51; A61K 31/7048

(52) U.S. Cl. CPC A61K 9/5153; B82Y 5/00; A61K 31/7048

(57) ABSTRACT

The present invention relates to polymeric nanoparticles comprising salinomycin and methods for treating certain diseases comprising administering these polymeric nanoparticles to a subject in need thereof.

### RELATED APPLICATION

This application claims the benefit of priority to U.S. Provisional Application No. 62/699,963, filed Jul. 18, 2018. The contents of this application are incorporated herein by reference in their entirety.

### FIELD

The present invention relates to the field of nanotechnology, in particular, to the use of biodegradable polymeric nanoparticles for the delivery of therapeutic agents such as salinomycin.

### BACKGROUND

Salinomycin, a monocarboxylic polyether antibiotic isolated from Streptomyces albus, has traditionally been used as an antibiotic. Salinomycin has recently been found to affect cancer cells and cancer stem cells in a number of ways, including causing cell cycle arrest, apoptosis, and overcoming multi-drug resistance. In vitro evidence has shown that salinomycin affects multiple cancer types including breast cancer, ovarian cancer, and pancreatic cancer. Treatment with salinomycin can result in toxicity, including neurotoxicity, and there remains a need to reduce such toxicity while still maintaining an effective dose of salinomycin.

### SUMMARY

The disclosure is based in part on the discovery that nanoparticles comprising salinomycin are less toxic when administered at the same dose than salinomycin alone in treating cancer. Accordingly, in one aspect, the invention provides a composition comprising: polymeric nanoparticles comprising a block copolymer comprising poly (lactic acid) (PLA) and poly (ethylene glycol) (PEG); and salinomycin.

The disclosure provides a composition comprising a polymeric nanoparticle comprising poly (lactic acid)-poly (ethylene glycol)-poly (propylene glycol)-poly (ethylene glycol) (PLA-PEG-PPG-PEG) tetra-block copolymer and salinomycin.

In various embodiments of the composition, the PLA-PEG-PPG-PEG tetra-block copolymer is formed from conjugation of PEG-PPG-PEG tri-block copolymer with PLA. For example, the conjugation is a chemical conjugation.

In another aspect, provided herein is a method of reducing proliferation, survival, migration, or colony formation ability of a rapidly proliferating cell in a subject in need thereof comprising contacting the cell with a therapeutically effective amount of a composition comprising polymeric nanoparticles comprising a poly(lactic acid)-poly(ethylene glycol)-poly(propylene glycol)-poly(ethylene glycol) (PLA-PEG-PPG-PEG) tetra block copolymer, and salinomycin, wherein the therapeutically effective amount is between about 0.025 mg of salinomycin per kg of mass of the subject (mg/kg) to about 5 mg/kg.

In some embodiments of the methods, the cell is a cancer cell. In another embodiment of the methods, the cell is a cancer stem cell.

In another aspect, provided herein is a method for treating cancer in a subject in need thereof comprising administering to the subject a therapeutically effective amount of a composition comprising polymeric nanoparticles comprising a poly(lactic acid)-poly(ethylene glycol)-poly(propylene glycol)-poly(ethylene glycol) (PLA-PEG-PPG-PEG) tetra block copolymer, and salinomycin; wherein the therapeutically effective amount is between about 0.025 mg/kg to about 5 mg/kg.

In some embodiments of the methods, the cancer is selected from the group consisting of breast cancer, ovarian cancer, pancreatic cancer, leukemia, lymphoma, osteosarcoma, gastric cancer, prostate cancer, colon cancer, lung cancer, liver cancer, kidney cancer, head and neck cancer, and cervical cancer. In an embodiment, the cancer is metastatic.

In another embodiment, the method further comprises administering an additional anti-cancer therapy to the subject. In an embodiment of the methods, the additional anti-cancer therapy is surgery, chemotherapy, radiation, hormone therapy, immunotherapy, or a combination thereof.

In some embodiments of the methods, the cancer is resistant or refractory to a chemotherapeutic agent.

In certain embodiments of the methods, the subject is a human.

In another aspect, provided herein is a method of reducing proliferation, survival, migration, or colony formation ability of cancer stem cells in a subject in need thereof comprising administering to the subject a therapeutically effective amount of a composition comprising polymeric nanoparticles comprising a poly(lactic acid)-poly(ethylene glycol)-poly(propylene glycol)-poly(ethylene glycol) (PLA-PEG-PPG-PEG) tetra block copolymer, and salinomycin, wherein the therapeutically effective amount is between about 0.025 mg/kg to about 5 mg/kg.

In embodiments of the methods, the therapeutically effective amount is between about 0.03 mg/kg and about 0.5 mg/kg.

In other embodiments of the methods, the therapeutically effective amount is between about 0.05 mg/kg and about 0.8 mg/kg.

In embodiments of the methods, the therapeutically effective amount is between about 0.08 mg/kg and about 1.1 mg/kg.

In embodiments of the methods, the composition is administered intravenously, intratumorally, or subcutaneously.

In some embodiments of the methods, the composition is administered at least once per day, once every other day, once per week, twice per week, once per month, or twice per month.

In an embodiment of the methods, the composition is administered once per week or twice per week for a duration of three weeks.

In an embodiment of the methods, the molecular weight of PLA is between about 10,000 and about 100,000 daltons.

In another embodiment of the methods, the molecular weight of PLA is between about 20,000 and 90,000 daltons.

In another embodiment of the methods, the molecular weight of PLA is between about 30,000 and 80,000 daltons.

In another embodiment of the methods, the molecular weight of PLA is between about 50,000 and 80,000 daltons.

In another embodiment of the methods, the molecular weight of PEG-PPG-PEG is between about 2,000 daltons and 18,000 daltons.

In another embodiment of the methods, the molecular weight of PEG-PPG-PEG is between about 10,000 daltons and 15,000 daltons.

In another embodiment of the methods, the molecular weight of PLA in the copolymer is 72,000 and the molecular weight of PEG-PPG-PEG is 12,500 daltons.

In another embodiment of the methods, the molecular weight of PLA in the copolymer is 35,000 and the molecular weight of PEG-PPG-PEG is 12,500 daltons.

In an embodiment of the methods, the composition further comprises a second therapeutic agent or a targeted anti-cancer agent.

In another embodiment of the methods, the molecular weight of PLA in the copolymer is 20,000 and the molecular weight of PEG-PPG-PEG is 2,000 daltons.

In another aspect, provided herein is a pharmaceutical composition comprising polymeric nanoparticles comprising a poly(lactic acid)-poly(ethylene glycol)-poly(propylene glycol)-poly(ethylene glycol) (PLA-PEG-PPG-PEG) tetra block copolymer, and salinomycin, and a pharmaceutically acceptable carrier.

In an embodiment of the pharmaceutical composition, the polymeric nanoparticle further comprises a targeting moiety attached to the outside of the polymeric nanoparticles.

In another aspect, provided herein is a dosage form comprising from about 12.5 mg to about 500 mg of the pharmaceutical composition comprising polymeric nanoparticles comprising a poly(lactic acid)-poly(ethylene glycol)-poly(propylene glycol)-poly(ethylene glycol) (PLA-PEG-PPG-PEG) tetra block copolymer, and salinomycin, and a pharmaceutically acceptable carrier.

In various embodiments of the composition, the molecular weight of PLA is between about 10,000 and about 100,000 daltons; between about 20,000 and 90,000 daltons; between about 30,000 and 80,000 daltons; between about 8,000 daltons and 18,000 daltons; or between about 10,000 daltons and 15,000. For example, the molecular weight of the PLA is about 10,000; 20,000; 30,000; 40,000; 50,000; 60,000; 70,000; 80,000; 90,000, or 100,000 daltons. In a further embodiment, the molecular weight of the PLA is about 12,500 daltons (i.e., 12.5 kDA) or about 72,000 daltons (i.e., 72 kDA). In an embodiment, the molecular weight of PEG-PPG-PEG from 2,000 to 12,5000 for generating the tetra block in an A-B structure, i.e., an alternating copolymer with regular alternating A and B subunits, is 12.5 kDa.

In various embodiments of the composition, the polymeric nanoparticles are formed of a polymer consisting essentially of poly(lactic acid)-poly(ethylene glycol) (PLA-PEG) di-block copolymer.

In various embodiments of the composition, the polymeric nanoparticles are formed of a polymer consisting essentially of poly(lactic acid)-poly(ethylene glycol)-poly(propylene glycol)-poly(ethylene glycol) (PLA-PEG-PPG-PEG) tetra-block copolymer.

In various embodiments of the composition, the polymeric nanoparticles further comprise a targeting moiety attached to the outside of the polymeric nanoparticles, and wherein the targeting moiety is an antibody, peptide, or aptamer. In various embodiments the targeting moiety comprises an immunoglobulin molecule, an scFv, a monoclonal antibody, a humanized antibody, a chimeric antibody, a humanized antibody, a Fab fragment, an Fab′ fragment, an F(ab′)2, an Fv, and a disulfide linked Fv.

In various embodiments of any of the compositions or methods provided herein, the nanoparticle is formed of the block copolymer comprising poly(lactic acid) (PLA) and poly(ethylene glycol) (PEG); and salinomycin. In an embodiment, the nanoparticle releases salinomycin over a period of time. In a further embodiment, the period of time is at least 1 day to 20 days. In various embodiments of the method, the period of time is about 5 days to 10 days.

In another aspect, provided herein is a pharmaceutical composition for use in reducing proliferation, survival, migration, or colony formation ability of a rapidly proliferating cell in a subject in need thereof, wherein the pharmaceutical composition comprises a poly(lactic acid)-poly(ethylene glycol)-poly(propylene glycol)-poly(ethylene glycol) (PLA-PEG-PPG-PEG) tetra block copolymer, and salinomycin wherein a therapeutically effective amount of the pharmaceutical composition is administered to the subject, and wherein the therapeutically effective amount is from about 0.025 mg/kg to about 5 mg/kg

In some embodiments of the pharmaceutical composition for use, the cell is a cancer cell. In another embodiment of the pharmaceutical composition for use, the cell is a cancer stem cell.

In another aspect, provided herein is a pharmaceutical composition for use in treating cancer in a subject in need thereof, wherein the pharmaceutical composition comprises a poly(lactic acid)-poly(ethylene glycol)-poly(propylene glycol)-poly(ethylene glycol) (PLA-PEG-PPG-PEG) tetra block copolymer, and salinomycin, wherein a therapeutically effective amount of the pharmaceutical composition is administered to the subject, and wherein the therapeutically effective amount is from about 0.025 mg/kg to about 5 mg/kg.

In some embodiments of the pharmaceutical composition for use, the cancer is selected from the group consisting of breast cancer, ovarian cancer, pancreatic cancer, leukemia, lymphoma, osteosarcoma, gastric cancer, prostate cancer, colon cancer, lung cancer, liver cancer, kidney cancer, head and neck cancer, and cervical cancer. In an embodiment, the cancer is metastatic.

In another embodiment, the pharmaceutical composition for use further comprises administering an additional anti-cancer therapy to the subject. In an embodiment of the pharmaceutical composition for use, the additional anti-cancer therapy is surgery, chemotherapy, radiation, hormone therapy, immunotherapy, or a combination thereof.

In some embodiments of the pharmaceutical composition for use, the cancer is resistant or refractory to a chemotherapeutic agent.

In certain embodiments of the pharmaceutical composition for use, the subject is a human.

In another aspect, provided herein is a pharmaceutical composition for use in reducing proliferation, survival, migration, or colony formation ability of cancer stem cells in a subject in need thereof, wherein the pharmaceutical composition comprises a poly(lactic acid)-poly(ethylene glycol)-poly(propylene glycol)-poly(ethylene glycol) (PLA-PEG-PPG-PEG) tetra block copolymer, and salinomycin, wherein a therapeutically effective amount of the pharmaceutical composition is administered to the subject, and wherein the therapeutically effective amount is from about 0.025 mg/kg to about 5 mg/kg.

In embodiments of the pharmaceutical composition for use, the therapeutically effective amount is between about 0.03 mg/kg and about 0.5 mg/kg.

In other embodiments of the pharmaceutical composition for use, the therapeutically effective amount is between about 0.05 mg/kg and about 0.8 mg/kg.

In embodiments of the pharmaceutical composition for use, the therapeutically effective amount is between about 0.08 mg/kg and about 1.1 mg/kg.

In embodiments of the pharmaceutical composition for use, the composition is administered intravenously, intratumorally, or subcutaneously.

In some embodiments of the pharmaceutical composition for use, the composition is administered at least once per day, once every other day, once per week, twice per week, once per month, or twice per month.

In an embodiment of the pharmaceutical composition for use, the composition is administered once per week or twice per week for a duration of three weeks.

In an embodiment of the pharmaceutical composition for use, the molecular weight of PLA is between about 10,000 and about 100,000 daltons.

In another embodiment of the pharmaceutical composition for use, the molecular weight of PLA is between about 20,000 and 90,000 daltons.

In another embodiment of the pharmaceutical composition for use, the molecular weight of PLA is between about 30,000 and 80,000 daltons.

In another embodiment of the pharmaceutical composition for use, the molecular weight of PLA is between about 50,000 and 80,000 daltons.

In another embodiment of the pharmaceutical composition for use, the molecular weight of PEG-PPG-PEG is between about 8,000 daltons and 18,000 daltons.

In another embodiment of the pharmaceutical composition for use, the molecular weight of PEG-PPG-PEG is between about 10,000 daltons and 15,000 daltons.

In another embodiment of the pharmaceutical composition for use, the molecular weight of PLA in the copolymer is 72,000 and the molecular weight of PEG-PPG-PEG is 12,500 daltons.

In another embodiment of the pharmaceutical composition for use, the molecular weight of PLA in the copolymer is 35,000 and the molecular weight of PEG-PPG-PEG is 12,500 daltons.

In an embodiment of the pharmaceutical composition for use, the composition further comprises a second therapeutic agent or a targeted anti-cancer agent.

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any two or more of the steps or features.

### BRIEF DESCRIPTION OF THE FIGURES

The following figures form part of the present specification and are included to further illustrate aspects of the present invention.

FIGS. 1A, 1B, and 1C are microscopic images of mouse liver sections stained with H&E showing a healthy liver section from a mouse in the control group (FIG. 1A), a mixture of fatty change and cytoplasmic glycogen from a mouse in the SAL 12.5 mg/kg group (FIG. 1B), and tension lipidosis from a mouse in the SAL 12.5 mg/kg group (FIG. 1C).

FIGS. 2A and 2B are microscopic images of mouse kidney sections stained with H&E showing a healthy kidney section with normal glomeruli (G), proximal (PT) and distal (DT) tubules from a mouse in the control group (FIG. 2A) and spacing of tubules (star) with atrophy of the lining epithelium, the reticulated casts within the lumina (arrows), and marked atrophy of renal corpuscle (black arrow) in a mouse from the 12.5 mg/kg SAL group (FIG. 2B).

FIGS. 3A and 3B are microscopic images of mouse testis sections stained with H&E showing healthy testis from a mouse from the control group (FIG. 3A) and shrunken seminiferous tubules and vacuolation in the germinal epithelium in a mouse from the 12.5 mg/kg SAL group (FIG. 3B).

FIGS. 4A and 4B are microscopic images of mouse epididymis sections stained with H&E showing healthy epididymis from a mouse from the control group (FIG. 4A) and disruption of epithelium with occurrence of vacuolization and necrotic cells in a mouse from the 12.5 mg/kg SAL group (FIG. 4B).

FIGS. 5A and 5B are electron micrographs of the salinomycin-nanoparticles. FIG. 5A shows a scanning electron micrograph of the salinomycin-nanoparticles. FIG. 5B shows a scanning electron micrograph of the salinomycin-nanoparticles. FIGS. 6A and 6B show the size distribution (FIG. 6A) and zeta potential (FIG. 6B) of the salinomycin-nanoparticles.

FIG. 7 is a graph showing the release of salinomycin from the salinomycin-nanoparticles.

FIG. 8 is a dose response curve of cell survival in H358 cells following treatment with salinomycin-nanoparticles.

FIGS. 9A and 9B are dose response curves of cell survival in NCI-H526 cells following treatment with salinomycin-nanoparticles (FIG. 9A). FIG. 9B is a dose response curve following treatment with two different formulations of salinomycin-nanoparticles.

FIG. 10 is a dose response curve of cell survival in NCI-H69 cells following treatment with salinomycin-nanoparticles.

FIG. 11 is a dose response curve of cell survival in MDA-MB-231 cells following treatment with salinomycin-nanoparticles.

FIG. 12 is a dose response curve of cell survival in SUM149 cells following treatment with salinomycin-nanoparticles.

FIG. 13 is a dose response curve of cell survival in MCF7 cells following treatment with salinomycin-nanoparticles.

FIG. 14 is a dose response curve of cell survival in MDA-MB-468 cells following treatment with salinomycin-nanoparticles.

FIGS. 15A and 15B are graphs showing tumor volume of H69 cells in mice (FIG. 15A) and body weight of the same mice (FIG. 15B) following treatment with salinomycin nanoparticles or vehicle control.

FIGS. 16A, 16B, 16C, 16D, and 16E are graphs showing the body weight and mortality of wild type mice following treatment with 5 mg/kg (FIG. 16A), 7.5 mg/kg (FIG. 16B), 10 mg/kg (FIG. 16C), 12.5 mg/kg (FIG. 16D), and 15 mg/kg (FIG. 16E) of salinomycin alone or salinomycin-nanoparticles.

FIGS. 17A, 17B and 17C are dose response curves showing the percentage inhibition of salinomycin (FIG. 17A), salinomycin nano-particle (FIG. 17B) on MDA-MB 231 cells in 3D anti-proliferation assays. FIG. 17C compares the data from FIG. 17A and FIG. 17B.

FIG. 18 shows pictures of cancer stem cells isolated from a TNBC patient and treated with PBS, salinomycin, salinomycin-NPs, or paclitaxel, along with the quantification of CD44+/CD24low cells.

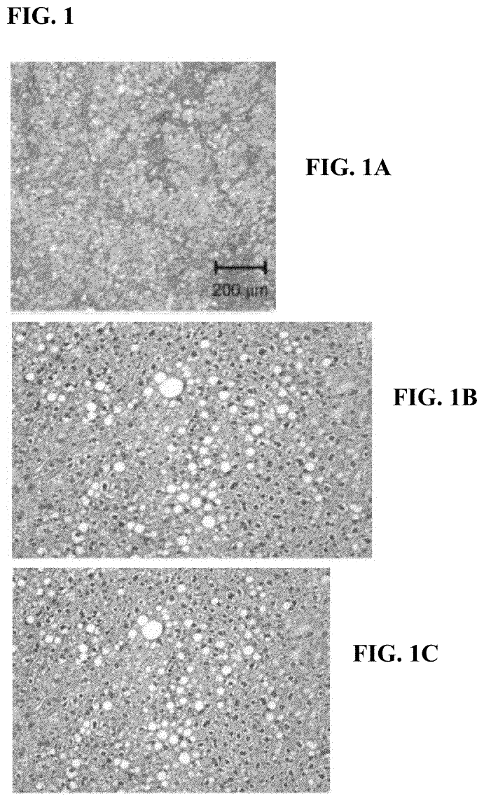

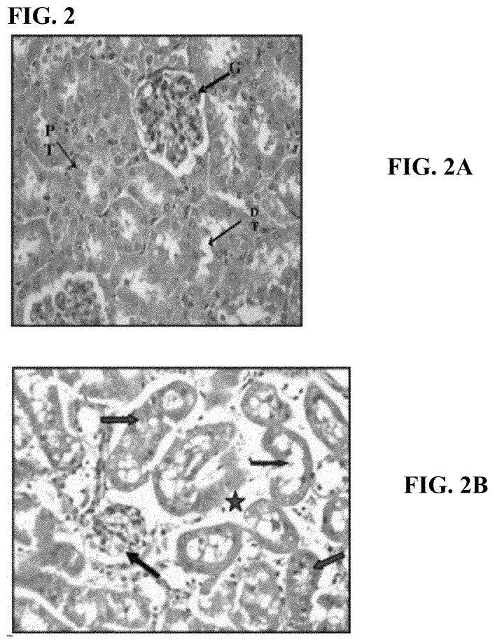

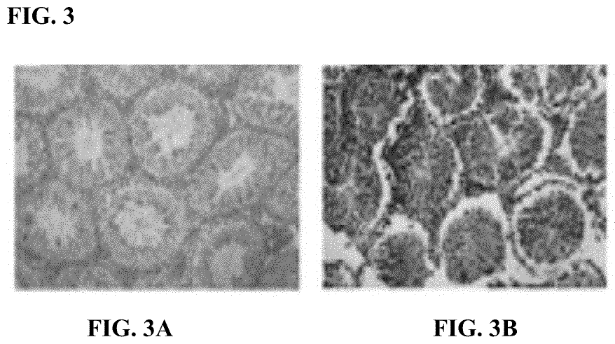

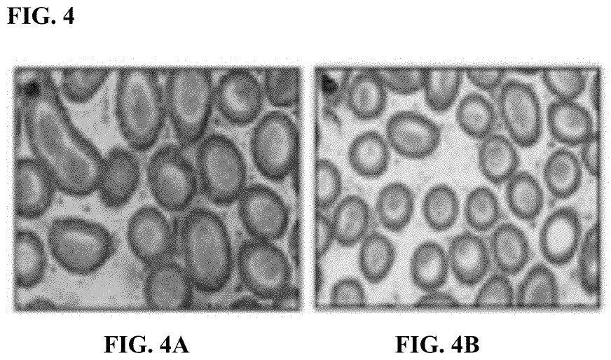

### DETAILED DESCRIPTION

The disclosure provides nanoparticles comprising salinomycin that are useful, inter alia, for treating or preventing cancers. The nanoparticles reduce the toxicity of salinomycin.

### Definitions

For convenience, before further description of the present invention, certain terms used in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The articles “a,” “an” and “the” are used to refer to one or to more than one (i.e., to at least one) of the grammatical object of the article.

The terms “comprise” “comprising” “including” “containing” “characterized by” and grammatical equivalents thereof are used in the inclusive, open sense, meaning that additional elements may be included. It is not intended to be construed as “consists of only.”

As used herein, “consisting of” and grammatical equivalent thereof exclude any element, step or ingredient not specified in the claim.

As used herein, the term “about” or “approximately” means within 5% of a given value or range.

The term “biodegradable” as used herein refers to both enzymatic and non-enzymatic breakdown or degradation of the polymeric structure.

The term “cationic” refers to any agent, composition, molecule or material that has a net positive charge or positive zeta potential under the respective environmental conditions. In various embodiments, nanoparticles described herein include a cationic polymer, peptide, protein carrier, or lipid.

As used herein, the term “multi-drug resistant” refers to cancer cells that have developed resistance to two or more chemotherapy drugs. Cancer cells can become multi-drug resistant by multiple mechanisms including decreased drug uptake and increased drug efflux.

As used herein, the term “resistant” or “refractive” to a therapeutic agent when referring to a cancer patient means that the cancer has innate, or achieved resistance to, the effects of the therapeutic agent as a result of contact with the therapeutic agent. Stated alternatively, the cancer is resistant to the ordinary standard of care associated with the particular therapeutic agent.

As used herein, the term “nanoparticle” refers to particles in the range between 10 nm to 1000 nm in diameter, wherein diameter refers to the diameter of a perfect sphere having the same volume as the particle. The term “nanoparticle” is used interchangeably as “nanoparticle(s)”. In some cases, the diameter of the particle is in the range of about 1-1000 nm, 10-500 nm, 20-300 nm, or 100-300 nm. In various embodiments, the diameter is about 30-170 nm. In certain embodiments, the diameter of the nanoparticle is about 1, 5, 10, 25, 50, 75, 100, 125, 150, 175, 200, 225, 250, 275, 300, 325, 350, 375,400, 425, 450, 475, 500, 525, 550, 575, 600, 625, 650, 675, 700, 725, 750, 775, 800, 825, 850, 875, 900, 925, 950, 975, or 1000 nm. In other embodiments, the diameter of the nanoparticle is 1, 5, 10, 25, 50, 75, 100, 125, 150, 175, 200, 225, 250, 275, 300, 325, 350, 375,400, 425, 450, 475, 500, 525, 550, 575, 600, 625, 650, 675, 700, 725, 750, 775, 800, 825, 850, 875, 900, 925, 950, 975, or 1000 nm.

In some cases, a population of particles may be present. As used herein, the diameter of the nanoparticles is an average of a distribution in a particular population.

As used herein, the term “polymer” is given its ordinary meaning as used in the art, i.e., a molecular structure comprising one or more repeat units (monomers), connected by covalent bonds. The repeat units may all be identical, or in some cases, there may be more than one type of repeat unit present within the polymer.

A “chemotherapeutic agent,” “therapeutic agent,” and “drug” is a biological (large molecule) or chemical (small molecule) compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, proteins, antibodies, photosensitizers, and kinase inhibitors. Chemotherapeutic agents include compounds used in “targeted therapy” and non-targeted, conventional chemotherapy.

A “targeting moiety” is a molecule that will bind selectively to the surface of targeted cells. For example, the targeting moiety may be a ligand that binds to the cell surface receptor found on a particular type of cell or expressed at a higher frequency on target cells than on other cells.

The targeting moiety or therapeutic agent can be a peptide or protein. “Proteins” and “peptides” are well-known terms in the art, and as used herein, these terms are given their ordinary meaning in the art. Generally, peptides are amino acid sequences of less than about 100 amino acids in length, and proteins are generally considered to be molecules of at least 100 amino acids. The amino acids can be in D- or L-configuration. A protein can be, for example, a protein drug, an antibody, a recombinant antibody, a recombinant protein, an enzyme, or the like. In some cases, one or more of the amino acids of the peptide or protein can be modified, for example by the addition of a chemical entity such as a carbohydrate group, a phosphate group, a farnesyl group, an isofarnesyl group, a fatty acid group, a linker for conjugation, functionalization, or other modification such as cyclization, by-cyclization and any of numerous other modifications intended to confer more advantageous properties on peptides and proteins. In other instances, one or more of the amino acids of the peptide or protein can be modified by substitution with one or more non-naturally occurring amino acids. The peptides or proteins may by selected from a combinatorial library such as a phage library, a yeast library, or an in vitro combinatorial library.

The term “combination,” “therapeutic combination,” or “pharmaceutical combination” as used herein refer to the combined administration of two or more therapeutic agents (e.g., co-delivery). Components of a combination therapy may be administered simultaneously or sequentially, i.e., at least one component of the combination is administered at a time temporally distinct from the other component(s). In embodiments, a component(s) is administered within one month, one week, 1-6 days, 18, 12, 10, 9, 8, 7, 6, 5, 4, 3, 2, 1 hour, or 30, 20, 15, 10, or 5 minutes of the other component(s).

The term “pharmaceutically acceptable” as used herein refers to those compounds, materials, compositions and/or dosage forms, which are, within the scope of sound medical judgment, suitable for contact with the tissues a warm-blooded animal, e.g., a mammal or human, without excessive toxicity, irritation allergic response and other problem complications commensurate with a reasonable benefit/risk ratio.

A “therapeutically effective amount” of a polymeric nanoparticle comprising one or more therapeutic agents is an amount sufficient to provide an observable or clinically significant improvement over the baseline clinically observable signs and symptoms of the disorders treated with the combination.

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from cancer.

The term “treating” or “treatment” as used herein comprises a treatment relieving, reducing or alleviating at least one symptom in a subject or producing a delay in the progression of a disease. For example, treatment can be the diminishment of one or several symptoms of a disorder or complete eradication of a disorder, such as cancer. Within the meaning of the present disclosure, the term “treat” also denotes to arrest and/or reduce the risk of worsening a disease. The term “prevent”, “preventing” or “prevention” as used herein comprises the prevention of at least one symptom associated with or caused by the state, disease or disorder being prevented.

As used herein, the term “human equivalent dose” refers to a dose of a composition to be administered to a human that is calculated from a specific dose used in an animal study.

As used herein, the term “rapidly proliferating cells” refers to cells having the capacity for autonomous growth (e.g., cancer cells).

As used herein, the term “cancer stem cell” refers to a cancer cell that has characteristics of a stem cell, such as giving rise to all cell types within a particular tumor type and the ability to self-renew. In some embodiments, the cancer stem cell is resistant or refractory to chemotherapy.

### Polymeric Nanoparticles Comprising Salinomycin

Provided herein are biodegradable polymeric nanoparticles for the delivery of salinomycin. Nanoparticles comprising salinomycin can be prepared using methods described in, e.g., US 2015-0353676 A1; PCT/US2016/060276 (published May 11, 2017); and PCT/US2017/059542, filed Nov. 1, 2017.

In an embodiment, the polymeric nanoparticles provided herein comprise a block copolymer comprising poly(lactic acid) (PLA) and poly(ethylene glycol) (PEG). Poly(lactic acid) (PLA), is a hydrophobic polymer, and is a preferred polymer for synthesis of the polymeric nanoparticles. However, poly(glycolic acid) (PGA) and block copolymer of poly lactic acid-co-glycolic acid (PLGA) may also be used. The hydrophobic polymer can also be biologically derived or a biopolymer. The molecular weight of the PLA used is generally in the range of about 2,000 g/mol to 80,000 g/mol. Thus, in an embodiment, the PLA used is in the range of about 10,000 g/mol to 80,000 g/mol. The average molecular weight of PLA may also be about 70,000 g/mol.

PEG is another preferred component to of the polymer used to form the polymeric nanoparticles as it imparts hydrophilicity, anti-phagocytosis against macrophage, and resistance to immunological recognition. Block copolymers like poly(ethylene glycol)-poly(propylene glycol)-poly(ethylene glycol) (PEG-PPG-PEG) are hydrophilic or hydrophilic-hydrophobic copolymers that can be used in the present invention. Block copolymers may have two, three, four, or more numbers of distinct blocks.

As used herein, one g/mole is equivalent to one “dalton” (i.e., dalton and g/mol are interchangeable when referring to the molecular weight of a polymer). “Kilodalton” as used herein refers to 1,000 daltons.

In a further embodiment, the polymeric nanoparticles provided herein comprise poly(lactic acid)-poly(ethylene glycol) (PLA-PEG) di-block copolymer.

In yet a further embodiment, the polymeric nanoparticles provided herein comprise poly(lactic acid)-poly(ethylene glycol)-poly(propylene glycol)-poly(ethylene glycol) (PLA-PEG-PPG-PEG) tetra-block copolymer. In various embodiments, the nanoparticles comprise a NANOPRO™, which is a biodegradable, long blood circulating, stealth, tetra-block polymeric nanoparticle platform (NanoProteagen Inc.; Massachusetts). The PLA-PEG-PPG-PEG tetra-block copolymer can be formed from chemical conjugation of PEG-PPG-PEG tri-block copolymer with PLA.

The synthesis and characterization of nanoparticles comprising poly(lactic acid)-poly(ethylene glycol)-poly(propylene glycol)-poly(ethylene glycol) (PLA-PEG-PPG-PEG) tetra block copolymer are described in PCT publication no. WO2013/160773, which is hereby incorporated by reference in its entirety. Polymeric nanoparticles comprising poly(lactic acid)-poly(ethylene glycol)-poly(propylene glycol)-poly(ethylene glycol) (PLA-PEG-PPG-PEG) tetra block copolymer have been shown to be safe, stable and non-toxic.

The process used to form this tetra-block copolymer comprises covalently attaching PEG-PPG-PEG to the poly-lactic acid (PLA) matrix, resulting in the block copolymer becoming a part of the matrix, i.e., a nanoparticle delivery system. This prevents leaching out of emulsifier into the medium.

In certain embodiments, molecular weight can be expressed as number average molecular weight or weight average molecular weight.

The number average molecular weight (Mn) is defined by:

$$Mn = \frac{{N}_{i} {M}_{i}}{{N}_{i}}$$

Where Mᵢ is the molecular weight of a chain and Nᵢ is the number of chains of that molecular weight. The weight average molecular weight (Mw) is defined by:

$$Mw = \frac{{N}_{i} {M}_{i}^{2}}{{N}_{i} {M}_{i}}$$

Compared to Mn, Mw takes into account the molecular weight of a chain in determining contributions to the molecular weight average. The more massive the chain, the more the chain contributes to Mw.

In some embodiments, the number average molecular weight (Mn) of the hydrophilic-hydrophobic block copolymer (e.g., PEG-PPG-PEG) is generally in the range of 1,000 to 20,000 g/mol. In a further embodiment, the average molecular weight (Mn) of the hydrophilic-hydrophobic block copolymer is about 4,000 g/mol to 15,000 g/mol. In some cases, the average molecular weight (Mn) of the hydrophilic-hydrophobic block copolymer is 4,400 g/mol, 8,400 g/mol, or 14,600 g/mol. In certain embodiments, the Mn of PEG-PPG-PEG is 1,100-15,000 g/mol, e.g., 4,000 to 13, 000 g/mol. In certain embodiments, the Mn of PEG-PPG-PEG is 10,000-13,000 g/mol. In other embodiments, the Mn of PEG-PPG-PEG is about 12,500 g/mol.

In some embodiments, a block copolymer of the instant invention consists essentially of a segment of poly(lactic acid) (PLA) and a segment of poly(ethylene glycol)-poly(propylene glycol)-poly(ethylene glycol) (PEG-PPG-PEG).

In an embodiment, a specific biodegradable polymeric nanoparticle is formed of the block copolymer poly(lactic acid)-poly(ethylene glycol)-poly(propylene glycol)-poly(ethylene glycol) (PLA-PEG-PPG-PEG).

Another specific biodegradable polymeric nanoparticle of the instant invention is formed of the block copolymer poly(lactic acid)-poly(ethylene glycol)-poly(propylene glycol)-poly(ethylene glycol)-poly(lactic acid) (PLA-PEG-PPG-PEG-PLA).

The biodegradable polymers of the instant invention can be formed by chemically modifying PLA with a hydrophilic-hydrophobic block copolymer using a covalent bond.

The biodegradable polymeric nanoparticles of the instant invention have, in various embodiments, a size in the range of about 1-1000 nm, a size in the range of about 30-300 nm, a size in the range of about 100-300 nm, or a size in the range of about 100-250 nm, or a size of at least about 100 nm.

The biodegradable polymeric nanoparticles of the instant invention have, in various embodiments, a size in the range of about 30-120 nm, a size of about 120-200 nm, or a size of about 200-260 nm, or a size of at least about 260 nm.

In an embodiment, the biodegradable polymer of the instant invention is substantially free of emulsifier, or may comprise external emulsifier by an amount of about 0.5% to 5% by weight.

In an embodiment, the biodegradable polymeric nanoparticle of the present invention is PLA-PEG-PPG-PEG, and the average molecular weight of the poly(lactic acid) block is about 60,000 g/mol, the average weight of the PEG-PPG-PEG block is about 8,400 or about 14,600 g/mol, and the external emulsifier is about 0.5% to 5% by weight.

In another embodiment, the biodegradable polymeric nanoparticle of the present invention is PLA-PEG-PPG-PEG, and the an average molecular weight of the poly(lactic acid) block is less than or equal to approximately 16,000 g/mol, the average weight of the PEG-PPG-PEG block is about 8,400 g/mol or about 14,600 g/mol, and wherein the composition is substantially free of emulsifier.

In an embodiment, the biodegradable polymeric nanoparticle is PLA-PEG-PPG-PEG, and the average molecular weight of the poly(lactic acid) block is between about 10,000 and about 100,000 daltons, between about 20,000 and 90,000 daltons, between about 30,000 and 80,000 daltons, between about 50,000 and 80,000 daltons, and about 72,000 daltons, the average weight of the PEG-PPG-PEG block is between about 8,000 daltons and 18,000 daltons, between about 12,000 daltons and 17,000 daltons and between about 8,400 or about 14,600 g/mol, and the external emulsifier is about 0.5% to 5% by weight.

In another embodiment, the biodegradable polymeric nanoparticle is PLA-PEG-PPG-PEG, and the an average molecular weight of the poly(lactic acid) block is less than or equal to approximately 100,000 daltons, the average weight of the PEG-PPG-PEG block is about 12,000 daltons or about 17,000 daltons, and wherein the composition is substantially free of emulsifier.

In another embodiment, the polymeric nanoparticles provided herein further comprise a cationic peptide.

In another aspect, provided herein is a polymeric nanoparticle formed of a polymer consisting essentially of a PLA-PEG-PPG-PEG tetra-block copolymer or PLA-PEG di-block copolymer, wherein the polymeric nanoparticles are loaded with salinomycin and, optionally, a second therapeutic agent.

Nanoparticles (also referred to herein as “NPs”) can be produced as nanocapsules or nanospheres. Salinomycin loading in the nanoparticle can be performed by either an adsorption process or an encapsulation process (Spada et al., 2011; Protein delivery of polymeric nanoparticles; World Academy of Science, Engineering and Technology: 76, incorporated herein, by reference, in its entirety). Nanoparticles, by using both passive and active targeting strategies, can enhance the intracellular concentration of drugs in cancer cells while avoiding toxicity in normal cells. When nanoparticles bind to specific receptors and enter the cell, they are usually enveloped by endosomes via receptor-mediated endocytosis, thereby bypassing the recognition of P-glycoprotein, one of the main drug resistance mechanisms (Cho et al., 2008, Therapeutic Nanoparticles for Drug Delivery in Cancer, Clin. Cancer Res.,2008, 14:1310-1316, incorporated herein, by reference, in its entirety). Nanoparticles are removed from the body by opsonization and phagocytosis (Sosnik et al., 2008; Polymeric Nanocarriers: New Endeavors for the Optimization of the Technological Aspects of Drugs; Recent Patents on Biomedical Engineering, 1: 43-59, incorporated herein, by reference, in its entirety). Nanocarrier based systems can be used for effective drug delivery with the advantages of improved intracellular penetration, localized delivery, protect drugs against premature degradation, controlled pharmacokinetic and drug tissue distribution profile, lower dose requirement and cost effectiveness (Farokhzad OC, et al.; Targeted nanoparticle-aptamer bioconjugates for cancer chemotherapy in vivo. Proc. Natl. Acad. Sci. USA 2006,103 (16): 6315-20; Fonseca C, et al., Paclitaxel-loaded PLGA nanoparticles: preparation, physicochemical characterization and in vitro anti-tumoral activity. J. Controlled Release 2002; 83 (2): 273-86; Hood et al., Nanomedicine, 2011, 6(7):1257-1272, incorporated herein, by reference, in their entireties).

The uptake of nanoparticles is indirectly proportional to their small dimensions. Due to their small size, the polymeric nanoparticles have been found to evade recognition and uptake by the reticulo-endothelial system (RES), and can thus circulate in the blood for an extended period (Borchard et al., 1996, Pharm. Res. 7: 1055-1058, incorporated herein, by reference, in its entirety). Nanoparticles are also able to extravasate at the pathological site like the leaky vasculature of a solid tumor, providing a passive targeting mechanism. Due to the higher surface area leading to faster solubilization rates, nano-sized structures usually show higher plasma concentrations and area under the curve (AUC) values. Lower particle size helps in evading the host defense mechanism and increase the blood circulation time. Nanoparticle size affects drug release. Larger particles have slower diffusion of drugs into the system. Smaller particles offer larger surface area but lead to fast drug release. Smaller particles tend to aggregate during storage and transportation of nanoparticle dispersions. Hence, a compromise between a small size and maximum stability of nanoparticles is desired. The size of nanoparticles used in a drug delivery system should be large enough to prevent their rapid leakage into blood capillaries but small enough to escape capture by fixed macrophages that are lodged in the reticuloendothelial system, such as the liver and spleen.

In addition to their size, the surface characteristics of nanoparticles are also an important factor in determining the life span and fate during circulation. Nanoparticles should ideally have a hydrophilic surface to escape macrophage capture. Nanoparticles formed from block copolymers with hydrophilic and hydrophobic domains meet these criteria. Controlled polymer degradation also allows for increased levels of agent delivery to a diseased state. Polymer degradation can also be affected by the particle size. Degradation rates increase with increase in particle size in vitro (Biopolymeric nanoparticles; Sundar et al., 2010, Science and Technology of Advanced Materials; doi:10.1088/1468-6996/11/1/014104, incorporated herein, by reference, in its entirety).

Poly(lactic acid) (PLA) has been approved by the US FDA for applications in tissue engineering, medical materials and drug carriers and poly(lactic acid)-poly(ethylene glycol) PLA-PEG based drug delivery systems are known in the art. US2006/0165987A1, incorporated herein, by reference, in its entirety, describes a stealthy polymeric biodegradable nanosphere comprising poly(ester)-poly(ethylene) multiblock copolymers and optional components for imparting rigidity to the nanospheres and incorporating pharmaceutical compounds. US2008/0081075A1, incorporated herein, by reference, in its entirety, discloses a novel mixed micelle structure with a functional inner core and hydrophilic outer shells, self-assembled from a graft macromolecule and one or more block copolymer. US2010/0004398A1, incorporated herein, by reference, in its entirety, describes a polymeric nanoparticle of shell/core configuration with an interphase region and a process for producing the same.

In various embodiments, the invention further comprises a cationic molecule that interacts with a therapeutic molecule to form a stable nanocomplex and/or serves as a cell penetrating peptide. In various embodiments, the cationic molecule cell comprises a penetrating peptide comprises or a protein transduction domain. In various embodiments, the cationic molecule is a cationic peptide that facilitates transduction of the therapeutic agent to the nucleus.

Provided herein are methods for preparing a polymeric nanoparticle comprising salinomycin and additional therapeutics. The resulting polymeric nanoparticle is not only non-toxic, safe, and biodegradable, but also stable in vivo with high storage stability, and can be safely used in a nanocarrier system or drug delivery system in the field of medicine. In embodiments, the polymeric nanoparticles provided herein can increase the half-life of the deliverable drug or therapeutic agent in vivo.

The preparation process can include providing salinomycin, dissolving a block polymer in a solvent to form a block copolymer solution; and adding the complex to the block copolymer solution to form a solution comprising the complex and the block copolymer.

In an embodiment, the block copolymer is PLA-PEG di-block copolymer. In an embodiment, the block copolymer is PLA-PEG-PPG-PEG tetra-block copolymer.

In an embodiment, the block copolymer solution is prepared at a concentration between about 2 mg/ml and 10 mg/ml. In a further embodiment, the block copolymer solution of is prepared at a concentration of about 6 mg/ml. In an embodiment, the process further comprises adding the solution comprising salinomycin to a solution comprising a surfactant. In a further embodiment, the solution resulting from combining salinomycin and the block polymer solution is stirred until stable nanoparticles are formed.

In various embodiments, the polymeric nanoparticles can adopt a non-spherical configuration upon swelling or shrinking.

The nanoparticle in various embodiments is amphiphilic in nature.

The zeta potential and PDI (Polydispersity Index) of the nanoparticles may be calculated (see U.S. Pat. No. 9,149,426, incorporated herein, by reference, in its entirety).

The polymeric nanoparticles have dimensions that may be measured using a Transmission Electron Microscope. In suitable embodiments, the diameter of the polymeric nanoparticles provided herein will be between about 100 and 350 nm in diameter or between about 100 and 30 nm in diameter or between about 100 and 250 nm. In a further embodiment, the diameter of the polymeric nanoparticles provided herein are about 100 nm, 110 nm, 120, nm, 130 nm, 140 nm, 150 nm, 160 nm, 170 nm, 180 nm, 190 nm, 200 nm, 210 nm, 220 nm, 230 nm, 240 nm, or 250 nm.

In an embodiment, the polymeric nanoparticles comprising a complex have a zeta-potential between about +5 to −90 mV, e.g., +4 to −75 mV, +3 to −30 mV, +2 to −25mV, +1 to −40 mV. In a further embodiment, the complex has a zeta-potential of about −30 mV.

Specific processes for polymeric nanoparticle formation and uses in pharmaceutical composition are provided herein for purpose of reference. These processes and uses may be carried out through a variety of methods apparent to those of skill in the art.

### Pharmaceutical Compositions

Also provided herein is a pharmaceutical composition comprising a salinomycin polymeric nanoparticle for use in medicine and in other fields that use a carrier system or a reservoir or depot of nanoparticles. The nanoparticles can be used in prognostic, therapeutic, diagnostic and/or theranostic compositions. Suitably, the nanoparticles of the present invention are used for drug and agent delivery (e.g., within a tumor cell), as well as for disease diagnosis and medical imaging in human and animals. Thus, the instant invention provides a method for the treatment of disease using the nanoparticles further comprising a therapeutic agent as described herein. The nanoparticles of the present invention can also be use in other applications such as chemical or biological reactions where a reservoir or depot is required, as biosensors, as agents for immobilized enzymes and the like.

Thus, in an aspect, provided herein is a pharmaceutical composition comprising

a) a polymeric nanoparticle comprising a block copolymer comprising poly(lactic acid) (PLA) and poly(ethylene glycol) (PEG); and

b) salinomycin.

In an embodiment, the polymeric nanoparticle comprises poly(lactic acid)-poly(ethylene glycol) (PLA-PEG) di-block copolymer.

In an embodiment, the polymeric nanoparticle comprises poly(lactic acid)-poly(ethylene glycol)-poly(propylene glycol)-poly(ethylene glycol) (PLA-PEG-PPG-PEG) tetra-block copolymer.

In a further embodiment, the PLA-PEG-PPG-PEG tetra-block copolymer is formed from chemical conjugation of PEG-PPG-PEG tri-block copolymer with PLA.

In an embodiment, the molecular weight of PLA is between about 10,000 and about 100,000 daltons.

In an embodiment of the compositions provided herein, the polymeric nanoparticles are formed of a polymer consisting essentially of poly(lactic acid)-poly(ethylene glycol) (PLA-PEG) di-block copolymer.

In an embodiment of the compositions provided herein, the polymeric nanoparticles are formed of a polymer consisting essentially of poly(lactic acid)-poly(ethylene glycol)-poly(propylene glycol)-poly(ethylene glycol) (PLA-PEG-PPG-PEG) tetra-block copolymer.

In an embodiment of the compositions provided herein, the polymeric nanoparticles further comprise a targeting moiety attached to the outside of the polymeric nanoparticles, and wherein the targeting moiety is an antibody, peptide, or aptamer.

Suitable pharmaceutical compositions or formulations can contain, for example, from about 0.1% to about 99.9%, preferably from about 1% to about 60%, of the active ingredient(s). Pharmaceutical formulations for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount may be reached by administration of a plurality of dosage units.

The pharmaceutical compositions can contain, as the active ingredient, one or more of nanoparticles in combination with one or more pharmaceutically acceptable carriers (excipients). In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Some examples of suitable excipients include lactose (e.g. lactose monohydrate), dextrose, sucrose, sorbitol, mannitol, starches (e.g. sodium starch glycolate), gum acacia, calcium phosphate, alginates, tragacanth, gelatin, calcium silicate, colloidal silicon dioxide, microcrystalline cellulose, polyvinylpyrrolidone (e.g. povidone), cellulose, water, syrup, methyl cellulose, and hydroxypropyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

### Methods of Treatment

The nanoparticles disclosed herein can be used to treat or prevent any condition or disorder which is known to or suspected of benefitting from treatment with salinomycin.

In one aspect, the salinomycin-containing nanoparticles are used to treat or prevent cancer or a precancerous condition. In some embodiments, the cancer is selected from the group consisting of breast cancer, ovarian cancer, pancreatic cancer, leukemia, lymphoma, osteosarcoma, gastric cancer, prostate cancer, colon cancer, lung cancer, liver cancer, kidney cancer, head and neck cancer, and cervical cancer.

In an embodiment, the cancer is breast cancer. In another embodiment, the breast cancer is triple negative breast cancer. In another embodiment, the breast cancer is hormone-dependent breast cancer.

In an embodiment, the cancer is lung cancer. In another embodiment, the lung cancer is non-small cell lung cancer. In another embodiment, the lung cancer is small cell lung cancer.

In one embodiment, the cancer is resistant or refractory to a chemotherapeutic agent. In another embodiment, the cancer is multi-drug resistant.

In an aspect, provided herein is a method for treating a disease in a subject in need thereof comprising administering to the subject a therapeutically effective amount of a pharmaceutical composition comprising a) a polymeric nanoparticle formed of a polymer comprising PLA-PEG di-block copolymer; and salinomycin.

In an embodiment of the methods provided herein, the pharmaceutical composition further comprises a chemotherapeutic agent or a targeted anti-cancer agent selected from the group consisting of lenalidomide, crizotinib, gleevec, herceptin, aystin, PD-1 checkpoint inhibitors, PDL-1 checkpoint inhibitors, CTLA-4 checkpoint inhibitors, doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, paclitaxel, and an indenoisoquinoline.

In an embodiment of the methods provided herein, the disease is cancer, an autoimmune disease, an inflammatory disease, a metabolic disorder, a developmental disorder, a cardiovascular disease, liver disease, an intestinal disease, an infectious disease, an endocrine disease and a neurological disorder.

In an embodiment of the methods provided herein, the nanoparticles are formed of a polymer consisting essentially of PLA-PEG di-block copolymer.

In an embodiment of the methods provided herein, the nanoparticles are formed of a polymer consisting essentially of PLA-PEG-PPG-PEG tetra-block copolymer.

In an embodiment, the polymeric nanoparticles are formed of a polymer consisting essentially of PLA-PEG di-block copolymer.

In an embodiment, the polymeric nanoparticles are formed of a polymer consisting essentially of PLA-PEG-PPG-PEG tetra-block copolymer.

The administration of a pharmaceutical composition provided herein may result not only in a beneficial effect with regard to alleviating, delaying progression of or inhibiting the symptoms of a disease or disorder, but also in further surprising beneficial effects, e.g. fewer side-effects, more durable response, an improved quality of life or a decreased morbidity, compared with, for example, delivering the agent without using the polymeric nanoparticle system described herein or by any other conventional means.

### Dosage and Administration

In one aspect, the present disclosure is directed to methods of treating cancer in a subject in need thereof, comprising administering to the subject a therapeutically effective amount of a composition comprising polymeric nanoparticles comprising a poly(lactic acid)-poly(ethylene glycol)-poly(propylene glycol)-poly(ethylene glycol) (PLA-PEG-PPG-PEG) tetra block copolymer, and salinomycin; wherein the therapeutically effective amount is between about 0.025 mg/kg to about 5 mg/kg.

In another aspect, provided herein is a method of reducing proliferation, survival, migration, or colony formation ability of cancer stem cells in a subject comprising administering to the subject a therapeutically effective amount of a composition comprising polymeric nanoparticles comprising a poly(lactic acid)-poly(ethylene glycol)-poly(propylene glycol)-poly(ethylene glycol) (PLA-PEG-PPG-PEG) tetra block copolymer, and salinomycin, wherein the therapeutically effective amount is between about 0.025 mg/kg to about 5 mg/kg.

In embodiments of the methods, the therapeutically effective amount is between about 0.1 mg/kg and about 2.5 mg/kg. In embodiments of the methods, the therapeutically effective amount is between about 0.5 mg/kg and about 5 mg/kg. In embodiments of the methods, the therapeutically effective amount is between about 1 mg/kg and about 5 mg/kg. In embodiments of the methods, the therapeutically effective amount is between about 2.5 mg/kg and about 5 mg/kg. In embodiments of the methods, the therapeutically effective amount is between about 0.025 mg/kg and about 0.5 mg/kg. In embodiments of the methods, the therapeutically effective amount is between about 0.025 mg/kg and about 0.1 mg/kg.

In other embodiments of the methods, the therapeutically effective amount is between about 0.025 mg/kg and about 1 mg/kg. In embodiments of the methods, the therapeutically effective amount is between about 1 mg/kg and about 2 mg/kg. In embodiments of the methods, the therapeutically effective amount is between about 2 mg/kg and about 3 mg/kg. In embodiments of the methods, the therapeutically effective amount is between about 3 mg/kg and about 4 mg/kg. In embodiments of the methods, the therapeutically effective amount is between about 4 mg/kg and about 5 mg/kg.

In embodiments of the methods, the therapeutically effective amount is between about 0.03 mg/kg and about 0.5 mg/kg. In an embodiment of the methods, the therapeutically effective amount is about 0.35 mg/kg. In an embodiment of the methods, the therapeutically effective amount is about 0.4 mg/kg. In other embodiments of the methods, the therapeutically effective amount is between about 0.05 mg/kg and about 0.8 mg/kg. In an embodiment of the methods, the therapeutically effective amount is about 0.61 mg/kg. In an embodiment of the methods, the therapeutically effective amount is about 0.69 mg/kg.

In embodiments of the methods, the therapeutically effective amount is between about 0.08 mg/kg and about 1.1 mg/kg. In an embodiment of the methods, the therapeutically effective amount is about 0.89 mg/kg. In an embodiment of the methods, the therapeutically effective amount is about 1.0 mg/kg.

In embodiments of the methods, the composition is administered intravenously, intratumorally, or subcutaneously.

In some embodiments of the methods, the composition is administered at least once per day, once every other day, once per week, twice per week, once per month, or twice per month. In an embodiment of the methods, the composition is administered at least once per day. In an embodiment of the methods, the composition is administered at least once every other day. In an embodiment of the methods, the composition is administered at least once per week. In an embodiment of the methods, the composition is administered at least twice per week. In an embodiment of the methods, the composition is administered at least once per month. In an embodiment of the methods, the composition is administered at least twice per month. In another embodiment, the composition is administered more than once per day.

In some embodiments of the methods, the composition is administered over a period of three weeks. In other embodiments of the methods, the composition is administered over a period of 30 days. In other embodiments of the methods, the composition is administered over a period of 60 days. In other embodiments of the methods, the composition is administered over a period of 90 days. In other embodiments of the methods, the composition is administered over a period of 120 days. In other embodiments of the methods, the composition is administered over a period of 150 days. In other embodiments of the methods, the composition is administered over a period of 6 months. In other embodiments of the methods, the composition is administered over a period of about 6 months to about 1 year. In other embodiments of the methods, the composition is administered over a period of about 1 year to about 2 years.

The methods and dosages disclosed herein have been found to reduce toxicity of salinomycin in vivo. Further, the compositions described herein allow salinomycin nanoparticles to be administered to a subject at a higher dose than salinomycin alone.

In certain embodiments, the therapeutically effective amount is a human equivalent dose that is determined from an animal experiment.

In an embodiment of the pharmaceutical composition, the polymeric nanoparticle further comprises a targeting moiety attached to the outside of the polymeric nanoparticles.

In another aspect, provided herein is a dosage form comprising from about 12.5 mg to about 500 mg of the pharmaceutical composition comprising polymeric nanoparticles comprising a poly(lactic acid)-poly(ethylene glycol)-poly(propylene glycol)-poly(ethylene glycol) (PLA-PEG-PPG-PEG) tetra block copolymer, and salinomycin, and a pharmaceutically acceptable carrier.

The effective dosage of the polymeric nanoparticles provided herein may vary depending on the particular protein, nucleic acid, and or other therapeutic agent used, the mode of administration, the condition being treated, and the severity of the condition being treated. Thus, the dosage regimen of the polymeric nanoparticle is selected in accordance with a variety of factors including the route of administration and the renal and hepatic function of the patient.

To determine efficacy, treatment may further comprise comparing one or more pre-treatment or post-treatment phenotypes to a standard phenotype. The standard phenotype is the corresponding phenotype in a reference cell or population of cells. Reference cells are one or more of the following, cells from a person or subject that is not suspected of having a protein degradation disorder, cells from the subject, cultured cells, cultured cells from the subject, or cells from the subject pre-treatment. Cells from the subject may include, for example, a bone marrow stromal cell, (BMSC), a peripheral blood mononuclear cell (PBMC), lymphocytes, hair follicles, blood cells, other epithelial cells, bone marrow plasma cells, primary cancer cells, patient derived tumor cells, normal or cancerous hematopoietic stem cells, neural stem cells, solid tumor cells, astrocytes, cancer stem cells, and the like.

### Combination Treatments

The compositions provided herein optionally further comprise an additional treatment modality, e.g., a therapeutic agent (e.g., a chemotherapeutic agent), radiation agent, hormonal agent, biological agent or an anti-inflammatory agent that is administered to a subject along with salinomycin.

Therapeutic agents that can be used in a combination therapy with salinomycin may include, e.g, lenalidomide, crizotinib or a histone deacetylase inhibitor (HDAC), such as those disclosed in U.S. Pat. No. 8,883,842, incorporated by reference, herein, in its entirety. Additional therapeutic agents include, e.g., gleevec, herceptin, aystin, PD-1 checkpoint inhibitors, PDL-1 checkpoint inhibitors, CTLA-4 checkpoint inhibitors, tamoxifen, trastuzamab, raloxifene, doxorubicin, fluorouracil/5-fu, pamidronate disodium, anastrozole, exemestane, cyclophos-phamide, epirubicin, letrozole, toremifene, fulvestrant, fluoxymester-one, trastuzumab, methotrexate, megastrol acetate, docetaxel, paclitaxel, testolactone, aziridine, vinblastine, capecitabine, goselerin acetate, zoledronic acid, taxol, vinblastine, and/or vincristine. Useful non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and mixtures thereof. For a more detailed description of the NSAIDs, see Paul A. Insel, Analgesic-Antipyretic and Antiinflammatory Agents and Drugs Employed in the Treatment of Gout, in Goodman & Gilman's The Pharmacological Basis of Therapeutics 617-57 Perry B. Molinhoff and Raymond W. Ruddon eds., 9^{th} ed 1996, Glen R. Hanson, Analgesic, Antipyretic and Anti-Inflammatory Drugs in Remington: The Science and Practice of Pharmacy Vol II 1196-1221 and A. R. Gennaro ed. 19^{th} ed. 1995 which are hereby incorporated by reference in their entireties.

In an embodiment, the additional chemotherapeutic agent or a targeted anti-cancer agent selected from the group consisting of doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, paclitaxel, and an indenoisoquinoline.

Although the subject matter has been described in considerable detail with reference to certain embodiments thereof, other embodiments are possible. As such, the spirit and scope of the appended claims should not be limited to the description of the specific embodiments contained therein.

### EXAMPLES

The disclosure will now be illustrated with working examples, and which is intended to illustrate the working of disclosure and not intended to restrictively any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1

### Preparation of Polymeric Nanoparticles of PLA-PEG-PPG-PEG Block Copolymer

Poly(lactic acid) (MW. −45,000-60,000 g/mol), PEG-PPG-PEG and tissue culture reagents were obtained from Sigma-Aldrich (St. Louis, Mo.). All reagents were analytical grade or above and used as received, unless otherwise stated. Cell lines were obtained from NCCS Pune, India or from ATCC, Maryland, USA

5 gm of poly (lactic acid) (PLA) with an average molecular weight of 60,000 g/mol was dissolved in 100 ml CH₂Cl₂ (dichloromethane) in a 250 ml round bottom flask. To this solution, 0.7 g of PEG-PPG-PEG polymer (molecular weight range of 1100-8400 Mn) was added. The solution was stirred for 10-12 hours at 0° C. To this reaction mixture, 5 ml of 1% N,N-dicyclohexylcarbodimide (DCC) solution was added followed by slow addition of 5 ml of 0.1% 4-Dimethylaminopyridine (DMAP) at −4° C. to 0° C/sub zero temperatures. There action mixture was stirred for the next 24 hours followed by precipitation of the PLA-PEG-PPG-PEG block copolymer with diethyl ether and filtration using Whatman filter paper No. 1. The PLA-PEG-PPG-PEG block copolymer precipitates so obtained were dried under low vacuum and stored at 2° C. to 8° C. until further use.

The PLA-PEG-PPG-PEG nanoparticles were prepared by an emulsion precipitation method. 100 mg of the PLA-PEG-PPG-PEG copolymer obtained by the above mentioned process was separately dissolved in an organic solvent, for example, acetonitrile, dimethyl formamide (DMF) or dichloromethane to obtain a polymeric solution.

The nanoparticles were prepared by adding this polymeric solution drop wise to the aqueous phase of 20 ml distilled water. The solution was stirred magnetically at room temperature for 10 to 12 hours to allow residual solvent evaporation and stabilization of the nanoparticles. The nanoparticles were then collected by centrifugation at 25,000 rpm for 10 min and washed thrice using distilled water. The nanoparticles were further lyophilized and stored at 2° C. to 8° C. until further use.

The shape of the nanoparticles obtained by the process mentioned above is essentially spherical. The particle size range was about 30 to 120 nm. The hydrodynamic radius of the nanoparticle was measured using a dynamic light scattering (DLS) instrument and is in the range of 110-120 nm.

### Example 2

### Preparation of a Salinomycin-Encapsulated Nanoparticle

The nanoparticles of the present invention are amphiphillic in nature and are capable of being loaded with both hydrophobic and hydrophilic drugs.

100 mg of the PLA-PEG-PPG-PEG nanoparticle prepared using the process of Example 1 is dissolved in 5 ml of an organic solvent like acetonitrile (CH₃CN), dimethyl formamide (DMF; C₃H₇NO), acetone or dichloromethane (CH₂Cl₂).

1-10 mg of salinomycin is dissolved in an aqueous solution and is added to the above polymeric solution. Salinomycin is usually taken in the weight range of about 10-20% weight of the polymer. This solution is briefly sonicated for 10-15 seconds at 250-400 rpm to produce a fine primary emulsion.

The fine primary emulsion is added drop wise using a syringe/micropipette to the aqueous phase of 20 ml distilled water containing F-127 poloxomer and stirred magnetically at 250 to 400 rpm at 25 ° C. to 30° C. for 10 to 12 hours in order to allow solvent evaporation and nanoparticle stabilization. The aqueous phase further comprises a sugar additive. The resulting nanoparticle suspension is allowed to stir overnight, in an open, uncovered condition to evaporate the residual organic solvent. The salinomycin encapsulated polymeric nanoparticles are collected by centrifugation at 10,000 g for 10 min or by ultrafiltration at 3000 g for 15 min. (Amicon Ultra, Ultracel membrane with 100,000 NMWL, Millipore, USA). The nanoparticles are resuspended in distilled water, washed thrice, and lyophilized. They are stored at 2° C. to 8° C. until further use. The polymeric nanoparticles are highly stable.

### Example 3

### Proof of Concept of Salinomycin-Nanoparticles (SAL-NPs) Preliminary Toxicity Study in CD2 Male Mice and Comparison Head-to-Head with Equal-Doses of SAL

A study was conducted in wild-type CD2 male mice to evaluate and compare the effects of salinomycin (SAL) at three different concentrations and compared to a formulation of SAL in biodegradable tetra-block polymeric nanoparticles.

MICE 20-25 g male CD2 mice between the ages 6 to 8 weeks from Taconic were used. The animals were acclimatized for five days before initiating the study.

Dose

Animals were injected intravenously either with 5.0 mg/kg, 8.5 mg/kg, or 12.5 mg/kg of SAL or SAL-NPs once, according to Table 1 below. Control animals were treated with PBS.

TABLE 1
Experimental design.

| Groups | Dose (mg/kg) | Number |
|---|---|---|
| A- SAL | 5 | 3 |
| B- SAL | 8.5 | 3 |
| C- SAL | 12.5 | 3 |
| D- SAL-NPs | 5 | 2 |
| E- SAL-NPs | 8.5 | 3 |
| F- SAL-NPs | 12.5 | 3 |
| G- Control | PBS | 3 |

Methods All animals were observed for changes (body weight, food and water intake) daily for seven days. Tolerability of the drug by animals was measured via clinical, weight and behavioral changes. The compounds were administered once on day 1. Seven days following administration, all animals were euthanized and blood was collected for a complete blood chemistry and hematology analysis (see Table 2 and 3 below). Post mortem was conducted to examine all of the animals. Different organs (brain, heart, lung, liver, spleen, stomach, intestine, kidney, and skin) were also isolated for histo-pathological evaluation by H & E staining.

TABLE 2
Complete blood chemistry analysis.

| | | Glucose | | Alk. | Blood | | Total | | | |
|---|---|---|---|---|---|---|---|---|---|---|
| | | mg/dl | Creatine | Phosphatase | urea N2 | cholesterol | protein | Albumin | Globulin | triglycerides |
| | | range170- | 0.1-0.4 | 30-110 | 18-30 | 110-190 | 5.0 to 6.5 | 3.0 to 4.2 | 1.8 to 2.5 | 50-160 |
| Group | n | 300 | mg/dl | mg/dl | mg/dl | mg/dl | g/dl | g/dl | g/dl | mg/dl |
| A-SAL | 1 | 236 | 0.14 | 65 | 21 | 123 | 5.7 | 3.7 | 1.94 | 68 |
| 5 mg/kg | 2 | 259 | 0.26 | 62 | 25 | 118 | 5.4 | 4.1 | 1.87 | 79 |
| | 3 | 278 | 0.12 | 78 | 21 | 121 | 5.9 | 3.4 | 2.19 | 76 |
| B-SAL | 1 | 264 | 0.21 | 82 | 27 | 124 | 5.4 | 3.2 | 2.08 | 69 |
| 8.5 mg/kg | 2 | 289 | 0.15 | 84 | 21 | 117 | 5.8 | 3.5 | 2.24 | 92 |
| | 3 | 235 | 0.13 | 65 | 20 | 119 | 5.7 | 4 | 1.93 | 79 |
| C-SAL | 1 | 264 | 0.17 | 47 | 19 | 114 | 5.1 | 3.8 | 2.35 | 86 |
| 12.5 mg/kg | 2 | 285 | 0.13 | 49 | 26 | 119 | 5.9 | 3.4 | 2.13 | 78 |
| | 3 | 271 | 0.15 | 73 | 22 | 132 | 6.2 | 3.8 | 1.94 | 93 |
| D-SAL-NP | 1 | 263 | 0.16 | 49 | 25 | 127 | 6.3 | 3.7 | 2.23 | 81 |
| 5 mg/kg | 2 | 287 | 0.2 | 53 | 24 | 118 | 5.8 | 4.1 | 1.89 | 56 |
| | 3 | | | | | | | | | |
| E-SAL-NP | 1 | 246 | 1.4 | 63 | 26 | 117 | 6.2 | 3.9 | 2.43 | 94 |
| 5 mg/kg | 2 | 294 | 0.12 | 54 | 21 | 114 | 5.6 | 3.2 | 2.31 | 89 |
| | 3 | 243 | 0.16 | 48 | 24 | 123 | 5.2 | 3.6 | 2.07 | 76 |
| F-SAL-NP | 1 | 256 | 0.12 | 38 | 28 | 132 | 5.4 | 3.8 | 2.11 | 67 |
| 5 mg/kg | 2 | 238 | 0.13 | 56 | 25 | 124 | 5.7 | 3.4 | 2.32 | 87 |
| | 3 | 228 | 0.11 | 73 | 26 | 126 | 6.2 | 3.1 | 1.95 | 94 |
| G-control | 1 | 254 | 0.23 | 69 | 27 | 118 | 6.1 | 3.4 | 2.05 | 85 |
| saline | 2 | 271 | 0.17 | 71 | 21 | 123 | 5.8 | 3.1 | 2.27 | 104 |
| | 3 | 246 | 0.13 | 58 | 24 | 127 | 5.6 | 3.5 | 2.13 | 76 |

TABLE 3
Hematology analysis.

| | | WBC | Neutro- | Lympho- | mono- | Eosino- | Baso- | RBC | Hemo- | He- | | Mean | Platelets |
|---|---|---|---|---|---|---|---|---|---|---|---|---|---|
| | | Range | phils | cytes | cytes | phils | phils | Range | globin | mat- | | Cell | Range |
| | | 10³//μl | Range | Range | Range | Range | Range | 10³//μl | Range | ocrit | MeanCell | He- | 10³//μl |
| | | 7.1 to | 10³//μl | 10³//μl | 10³//μl | 10³//μl | 10³//μl | 8.4 to | g/dl | % 47 | Volume | moglobin | 780 to |
| Group | n | 17.2 | 0.99 to 6.2 | 5 to 11.5 | .09 to 0.63 | 0 to 0.75 | 0 to 0 | 10.5 | 12.5 to 15.9 | to 39 | 42 to 48 | 14-17 | 1800 |
| A-SAL | 1 | 9.72 | 3.56 | 8.95 | 0.21 | 0.17 | 0 | 9.34 | 14.32 | 41 | 46 | 17 | 1237 |
| 5 mg/kg | 2 | 10.43 | 4.92 | 9.14 | 0.34 | 0.21 | 0 | 8.76 | 13.96 | 43 | 42 | 17 | 1465 |
| | 3 | 10.89 | 4.17 | 10.04 | 0.41 | 0.15 | 0 | 9.16 | 14.08 | 39 | 45 | 15 | 1587 |
| B-SAL | 1 | 12.83 | 3.59 | 7.38 | 0.32 | 0.06 | 0 | 8.93 | 12.96 | 44 | 43 | 16 | 1549 |
| 8.5 mg/kg | 2 | 10.94 | 4.87 | 9.12 | 0.42 | 0.12 | 0 | 9.17 | 13.48 | 41 | 45 | 15 | 1328 |
| | 3 | 1.24 | 5.01 | 8.04 | 0.37 | 0.09 | 0 | 9.23 | 14.76 | 47 | 47 | 15 | 1269 |
| C-SAL | 1 | 14.23 | 4.83 | 7.93 | 0.28 | 0.13 | 0 | 9.78 | 15.37 | 43 | 44 | 14 | 1652 |
| 12.5 | 2 | 14.72 | 5.62 | 8.65 | 0.25 | 0.18 | 0 | 9.15 | 14.86 | 42 | 46 | 15 | 1438 |
| mg/kg | 3 | 15.29 | 5.09 | 8.13 | 0.34 | 0.06 | 0 | 8.93 | 14.29 | 45 | 43 | 14 | 1394 |
| D-SAL-NP | 1 | 13.58 | 3.97 | 9.75 | 0.42 | 0.08 | 0 | 9.18 | 15.18 | 42 | 45 | 14 | 1372 |
| 5 mg/kg | 2 | 12.35 | 4.82 | 8.32 | 0.29 | 0.11 | 0 | 10.02 | 13.17 | 40 | 48 | 15 | 1298 |
| | 3 | | | | | | | | | | | | |
| E-SAL-NP | 1 | 11.97 | 5.24 | 7.35 | 0.4 | 0.14 | 0 | 8.98 | 14.23 | 41 | 46 | 16 | 1346 |
| 5 mg/kg | 2 | 14.26 | 4.87 | 9.18 | 0.33 | 0.05 | 0 | 10.14 | 15.24 | 43 | 45 | 17 | 1487 |
| | 3 | 11.93 | 4.58 | 9.33 | 0.28 | 0.07 | 0 | 9.32 | 13.37 | 45 | 43 | 15 | 1582 |
| F-SAL-NP | 1 | 12.57 | 5.18 | 8.27 | 0.36 | 0.14 | 0 | 9.84 | 14.09 | 43 | 47 | 15 | 1423 |
| 5 mg/kg | 2 | 11.74 | 3.99 | 7.65 | 0.34 | 0.12 | 0 | 9.27 | 13.88 | 47 | 45 | 14 | 1561 |
| | 3 | 10.95 | 4.76 | 6.89 | 0.45 | 0.04 | 0 | 10.17 | 14.21 | 44 | 43 | 15 | 1672 |
| G-control | 1 | 12.46 | 5.25 | 7.28 | 0.32 | 0.08 | 0 | 8.94 | 15.12 | 42 | 44 | 17 | 1348 |
| saline | 2 | 11.92 | 5.72 | 9.34 | 0.45 | 0.03 | 0 | 10.23 | 14.19 | 44 | 46 | 14 | 1732 |
| | 3 | 10.87 | 4.36 | 8.17 | 0.28 | 0.07 | 0 | 8.56 | 13.27 | 41 | 47 | 15 | 1436 |

Results

Each experimental animal was individually and closely observed for any clinical and behavioral changes, including their water and food intake, urination, defecation and any other observable changes in the course of the study.

There was observable change in body weight of the group that received 12.5 mg/kg SAL. Moreover, slight reduction in weight was also observed in a group of animals dosed with 8.5 mg/kg SAL. No observable changes were there in the 5 mg/kg SAL group. Interestingly, there were no observable changes in the body weight of animals in the groups where SAL-NPs were administered at all doses. Food and water intake were at a constant level for all of the groups, except on the 7th day for the 12.5 mg/kg SAL group. See Table 4 below for body weight data and Table 5 for post mortem findings.

TABLE 4
Body weight observations.

| | | 12- | 13- | 14- | 15- | 16- | 17- | 18- | 19- |
|---|---|---|---|---|---|---|---|---|---|
| Group | n | Mar | Mar | Mar | Mar | Mar | Mar | Mar | Mar |
| A-SAL | 1 | 23.7 | 23.4 | 23.4 | 23.5 | 23.3 | 23.6 | 23.6 | 23.9 |
| 5 mg/kg | 2 | 24.1 | 24 | 23.9 | 24.2 | 24.1 | 24.4 | 24.3 | 24.7 |
| | 3 | 24.2 | 24 | 24 | 24.3 | 24.3 | 24.5 | 24.8 | 24.9 |
| B-SAL | 1 | 24.7 | 24.2 | 24.1 | 24.5 | 24.4 | 24.7 | 24.5 | 24.7 |
| 8.5 mg/kg | 2 | 24.2 | 23.9 | 24 | 24.3 | 24.3 | 24.5 | 24.4 | 24.6 |
| | 3 | 23.6 | 23.4 | 23.3 | 23.4 | 23.6 | 23.7 | 23.8 | 24 |
| C-SAL | 1 | 24.1 | 23.7 | 23.5 | 23.3 | 23 | 23 | 22.9 | 22.7 |
| 12.5 mg/kg | 2 | 24.5 | 24.2 | 24.1 | 14 | 23.9 | 24 | 23.8 | 23.5 |
| | 3 | 24.7 | 24.1 | 24 | 23.9 | 23.7 | 23.4 | 23.5 | 23.3 |
| D-SAL-NP | 1 | 24.2 | 24 | 24.1 | 24.3 | 24.2 | 24.6 | 24.6 | 24.8 |
| 5 mg/kg | 2 | 23.6 | 23.5 | 23.7 | 23.8 | 23.9 | 24 | 24.1 | 24.5 |
| | 3 | | | | | | | | |
| E-SAL-NP | 1 | 24.6 | 24.4 | 24.3 | 24.6 | 24.7 | 24.8 | 24.9 | 25.1 |
| 5 mg/kg | 2 | 23.8 | 23.5 | 23.6 | 23.9 | 23.9 | 24.1 | 24.2 | 24.3 |
| | 3 | 24 | 23.7 | 23.9 | 24.1 | 24.4 | 24.5 | 24.7 | 24.9 |
| F-SAL-NP | 1 | 24.6 | 24.4 | 24.3 | 24.1 | 24.3 | 24.3 | 24.5 | 24.7 |
| 5 mg/kg | 2 | 24.2 | 23.9 | 23.9 | 24.1 | 24.3 | 24.2 | 24.4 | 24.4 |
| | 3 | 23.9 | 23.7 | 23.5 | 23.7 | 23.9 | 24 | 24.1 | 24.2 |
| G-control | 1 | 23.7 | 23.9 | 23.7 | 24.1 | 24.3 | 24.2 | 24.6 | |
| saline | 2 | 24.1 | 24.5 | 24.3 | 24.5 | 24.7 | 24.8 | 24.7 | |
| | 3 | 23.9 | 23.7 | 23.9 | 24.3 | 24.1 | 24.5 | 24.6 | |

TABLE 5
Post mortem observations.

| Group | n | Post-mortem findings | Testis Wt | Trauma | Hind Leg |
|---|---|---|---|---|---|
| A-SAL | 1 | No Changes observed | 3.65 | | |
| 5 mg/kg | 2 | No Changes observed | 4.27 | | |
| | 3 | No Changes observed | 3.92 | | |
| B-SAL | 1 | No Changes observed | 2.71 | | |
| 8.5 mg/kg | 2 | Slight enlargement of | 2.89 | No trauma | Hind leg |
| | | testis with redness | | | lagging 3/18 |
| | 3 | No Changes observed | 2.86 | | |
| C-SAL | 1 | Testis enlarged, red in | 2.14 | No Trauma | Hind leg |
| 12.5 mg/kg | | color, inflammation. | | noted | lagging 3/17 |
| | | Epidydimus enalrged | | | |
| | 2 | Testis enlarged, red in | 1.09 | No Trauma | Hind leg |
| | | color, inflammation. | | noted | lagging 3/18 |
| | | Epidydimus enalrged | | | |
| | 3 | Testis enlarged, red in | 2.32 | No Trauma | Hind leg |
| | | color, inflammation. | | noted | lagging 3/18 |
| | | Epidydimus enalrged | | | |
| D-SAL-NP | 1 | No changes observed | 3.47 | | |
| 5 mg/kg | 2 | No changes observed | 4.69 | | |
| | 3 | | | | |
| E-SAL-NP | 1 | No changes observed | 4.18 | | |
| 8.5 mg/kg | 2 | No changes observed | 3.96 | | |
| | 3 | No changes observed | 4.23 | | |
| F-SAL-NP | 1 | No changes observed | 4.62 | | |
| 12.5 mg/kg | 2 | No changes observed | 3.87 | | |
| | 3 | No changes observed | 4.06 | | |
| G-control saline | 1 | | 4.18 | | |
| | 2 | | 4.32 | | |
| | 3 | | 4.19 | | |

No changes in urination and defecation were observed in animals from all the groups. Slight rough coat was observed on the 6t^{h} day on animal numbers 2 and 3 in the 12.5 mg/kg SAL group. Roughness of the fur was slightly worsened on day 7 in this group of animals.

There was a clear sign of hind leg lagging and decreased movement on day 6 in animal number 1 in the 12.5 mg/kg SAL group, which worsened on day 7. This lagging of the hind limbs was also seen in animals 2 and 3 (SAL group of 12.5 mg/kg) on day 7. Hind leg lagging was seen to a lesser extent in the SAL 8.5 mg/kg group. All animals in the 12.5 mg/kg SAL, 8.5 mg/kg SAL and 5 mg/kg SAL groups were lethargic in movement.

Importantly, none the animals in the groups that received SAL-NPs showed any symptoms of hind leg lagging and had no impairment of movement. This data shows that the formulation of nanoparticles and salinomycin is surprisingly less toxic than salinomycin alone.

On day 7 of the study, a post mortem inspection was conducted on each and every animal. The group that received 5 mg/kg SAL did not show any observable changes under a dissection microscope in any of the 11 organs examined. Animal number 2 of the 8.5 mg/kg SAL group had a slightly reddish and slightly enlarged testis. All the animals in this group had reduced testis weight as compared to the normal animals. There was slight enlargement observed on the fascia around the testis. No trauma was observed and the epididymis was found to be free of any changes. All the animals in the group that received 12.5 mg/kg SAL had reduction in the weight of testis and epididymis, reddening of the fascia surrounding testis, thereby looking as if it is enlarged. None of these animals showed any signs of trauma. All the animals in the groups that received SAL-NPs (5 mg/kg, 8.5 mg/kg and 12.5 mg/kg) did not show any such changes and appeared normal.

All the animals did well in the study, except the Salinomycin 12.5 mg/kg group. The histopathological studies were conducted on brain, heart, lung, liver, spleen, stomach, intestine, kidney, muscles, and skin tissues by H & E staining. The tissues were collected on the day 7 and passed through ascending concentration of alcohol, and then preserved in Buoyins Solution for sectioning.

On microscopical examination of all the tissues, no changes were observed in the brain, heart, lung, spleen, stomach, testis, epididymis, sciatic nerves, intestine, muscles and skin of all the animals in the study that received SAL-NPs. However, changes were observed in the kidney, liver, testis and epididymis of the groups that received 12.5 mg/kg of SAL.

There was mixture of fatty change, cytoplasmic glycogen and tension lipidosis in the livers of animals that received 12.5 mg/kg SAL. These changes were seen in all three animals at the 12.5 mg/kg SAL dose. No changes in the liver were observed in animals that received 12.5 mg/kg SAL-NPs.

The testis of all three animals that received 12.5 mg/kg of SAL had the seminiferous tubules shrunken and vacuolation in the germinal epithelium. The disruption of epithelium with occurrence of vacuolization and necrotic cells were observed in the cross section of epididymis in animals that received 12.5 mg/kg SAL. The treatment induced various structural changes (shrinkage) in the seminiferous tubules and interstitium of the testis. Epithelial gaps, epithelial sloughing and germ cell degeneration were also observed. Surprisingly, the animals treated with 12.5 mg/kg of SAL-NPs had no changes in their epididymis.

All three animals dosed with 12.5 mg/kg SAL showed spacing of tubules with atrophy of the lining epithelium of the kidney. The reticulated casts within their lumina was observed and there were marked atrophy of renal corpuscle. The animals treated with 12.5 mg/kg of SAL-NPs had no changes in the kidneys.

Conclusion

There is clear indication that the animals treated with SAL had toxicity to the liver, kidney, testis, and epididymis, whereas there were no changes observed even at the highest concentration of the SAL-NPs groups. This study indicates that all three concentrations of the SAL-NPs were well tolerated by the animals.

### Example 4

### Calculation of the Human Equivalent Dose (HED) of Salinomycin-Nanoparticles (SAL-NPs)

The human equivalent doses (HEDs) of the salinomycin-nanoparticle (SAL-NP) doses used in the mouse study were calculated by two different equations as disclosed in Nair and Jacob, “A simple practice guide for dose conversion between animals and human” (2016) and J. Basic Clin. Pharma. 27-31; and also disclosed in the FDA's “Guidance for Industry” (July 2005), incorporated, herein, by reference in their entireties. Specific embodiments of HEDs for SAL-NPs are disclosed in Table 6 below.

TABLE 6
HEDs of SAL-NP doses used in Example 3.

| Mouse dose | HED Equation 1 | HED Equation 2 |
|---|---|---|
| (mg/kg) | (mg/kg) | (mg/kg) |
| 5 | 0.35 | 0.4 |
| 8.5 | 0.61 | 0.69 |
| 12.5 | 0.89 | 1.01 |

The disclosures of each and every patent, patent application, and publication cited herein are hereby incorporated herein by reference in their entirety.

While the invention has been disclosed with reference to specific embodiments, it is apparent that other embodiments and variations of this invention can be devised by others skilled in the art without departing from the true spirit and scope of the invention. The appended claims are intended to be construed to include all such embodiments and equivalent variations.

### Example 5

### Effect of Salinomycin-Containing Nanoparticles on Cell Survival of Multiple Cancer Cell Lines

The effect of salinomycin-containing nanoparticles on cancer cell survival was assessed using the Alamar Blue assay. Based on the growth rate of each cell line, 1500 to 4000 cells/well were plated in 96 well plates and allowed to grow overnight at 37° C., 5% CO₂. Cells were treated with different concentrations of salinomycin-containing nanoparticles for five days with three-fold serial dilutions for eight concentrations.

Alamar Blue reagent (1:10 dilution in the culture medium) was then added to the wells and incubated for 2-4 hrs. The change in absorption was measured with excitation at 570 nM and emission at 600 nM. The percentage survival was calculated compared to the untreated control as 100%.

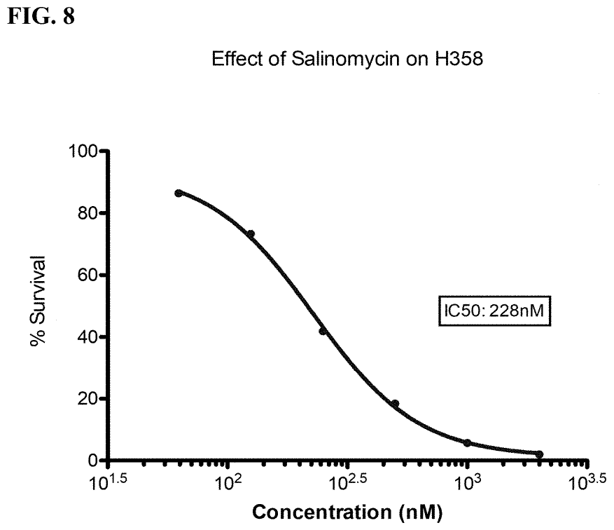

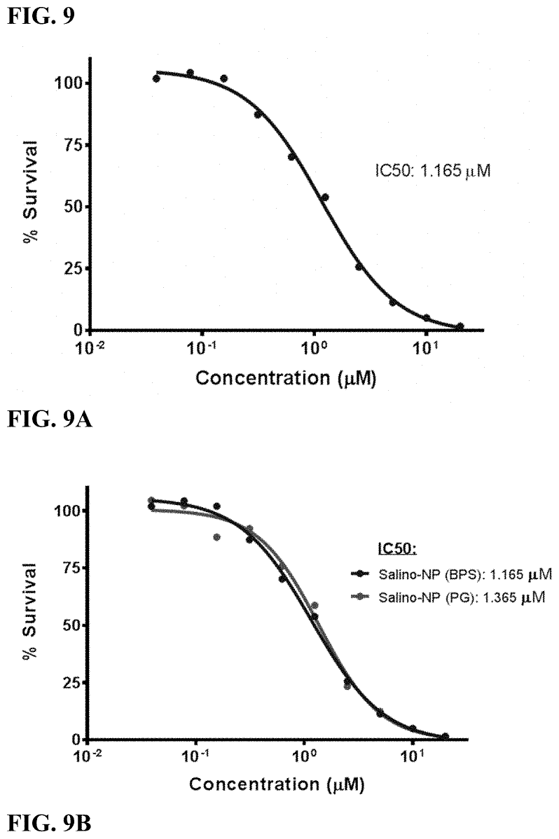

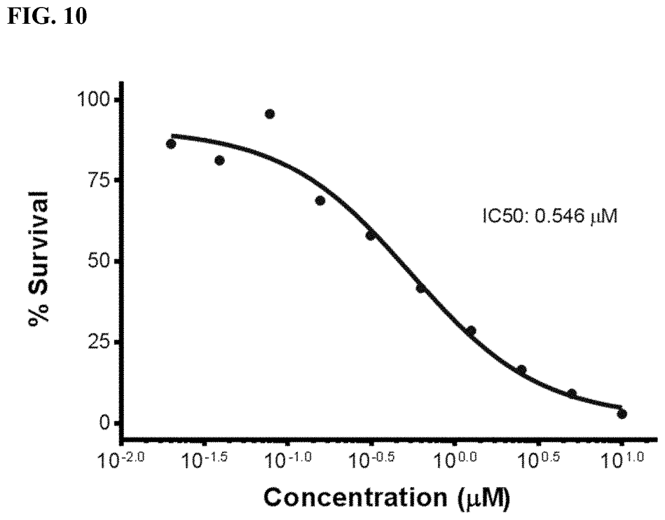

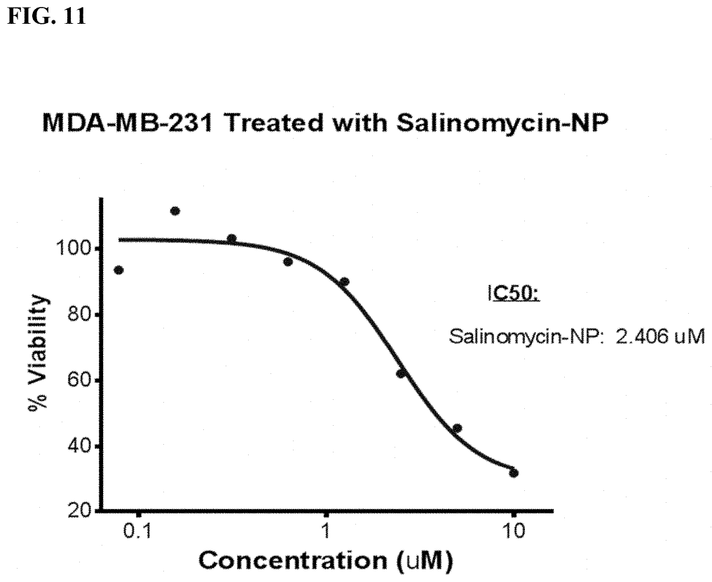

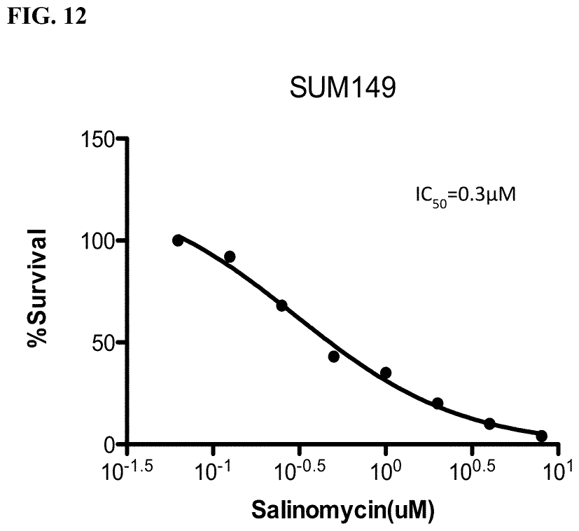

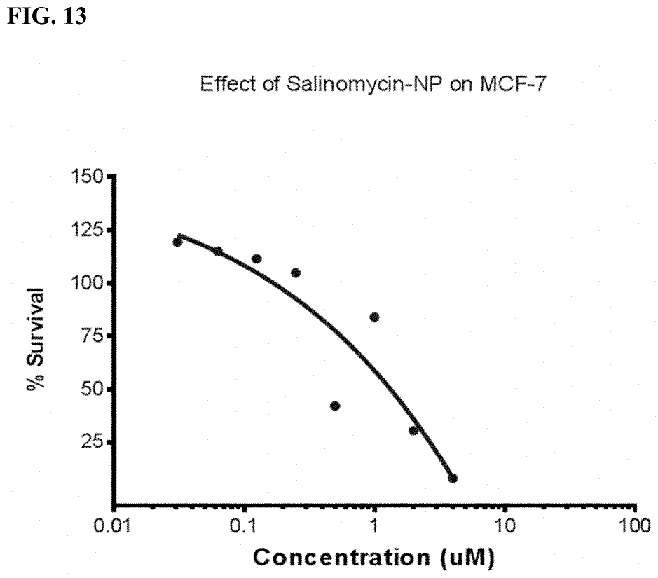

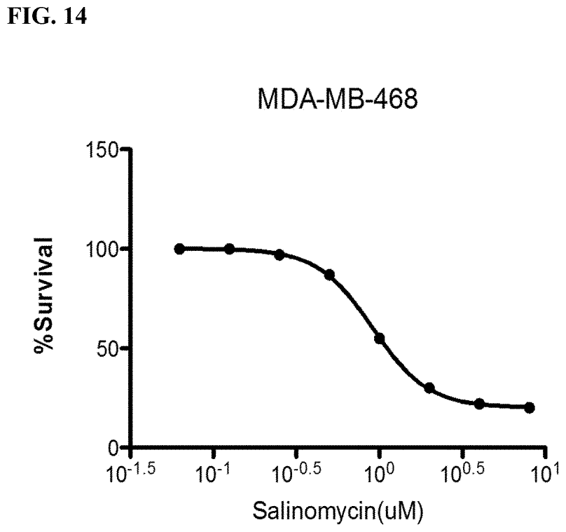

The results of Bronchioalveolar carcinoma (non-small cell lung cancer) cell lines are shown in FIG. 8 (NCI-H358). The results of small cell lung cancer cell lines are shown in FIG. 9A (NCI-H526), FIG. 9B (NCI-H526, two different formulations of SAL-NPs) and FIG. 10 (NCI-H69). The results of triple negative breast cancer cell lines are shown in FIG. 11 (MDA-MB-231), FIG. 12 (SUM149), and FIG. 14 (MDA-MB-468). The results for hormone-dependent breast carcinoma cells are shown in FIG. 13 (MCF-7). Shown in all graphs is the percent survival (y-axis) as a function of nanoparticle concentration (x-axis). The IC₅₀ value of each cell line was calculated from the cell survival data (see Table 7 below).

TABLE 7
IC₅₀ values of SAL-NPs in cancer cell lines

| Cell line | IC₅₀ (μM) |
|---|---|
| NCI-H358 | 0.228 |
| NCI-H526 | 1.165 |
| NCI-H69 | 0.546 |
| MDA-MB-231 | 2.406 |
| SUM149 | 0.3 |
| MCF-7 | 1.5 |
| MDA-MB-468 | 0.91 |

### Example 6

### Effect of Salinomycin-Containing Nanoparticles on Mammospheres

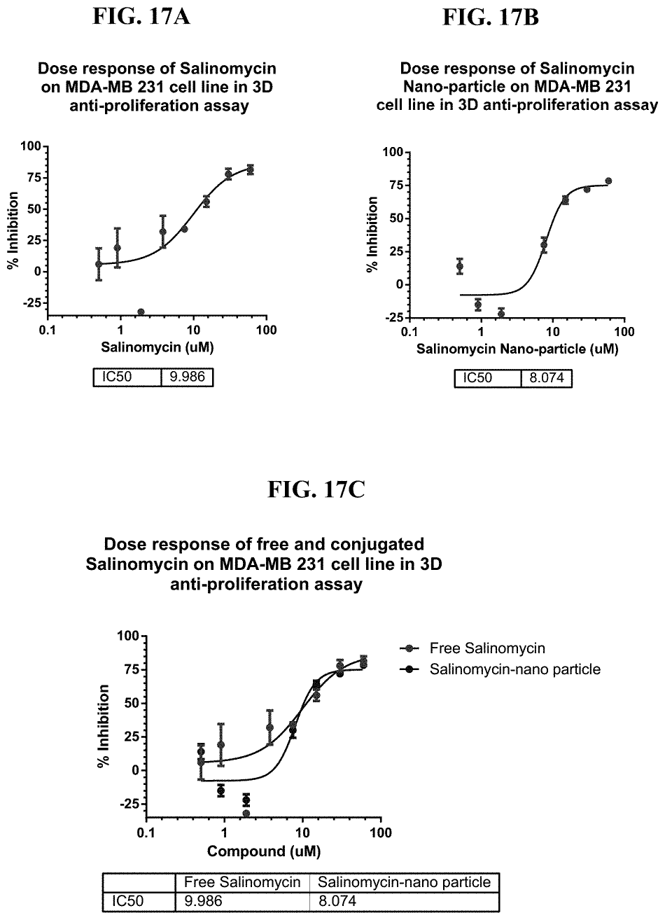

Cancer stem cell-mediated mammospheres were generated in serum-free tumorosphere growing special media from MDA-MB-231 Triple Negative Breast Cancer (TNBC) cells as 3D cultures. Following successful generation, mammospheres were treated with eight different concentrations of salinomycin or salinomycin-NPs using duplicate wells for 72 hours. Following incubation, WST-1 reagent was added and the plates were incubated for an additional 60 minutes and read for luminescence or absorbance at 630 nm. The results of the anti-proliferation assays in 3D Mammospheres with SAL and SAL-NPs are shown in FIG. 17A-17C.

### Example 7

### Effect of Salinomycin-Containing Nanoparticles on Cancer Stem Cells Isolated from a TNBC Patient

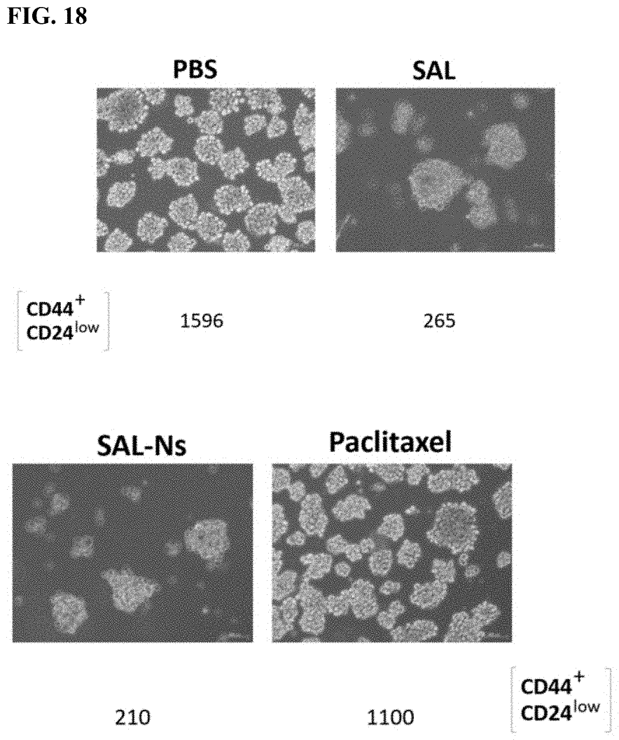

Flow cytometric identification of cancer stem cells (CSCs) by CD24-PE & CD44-FITC antibody staining in triple negative breast cancer (TNBC) patient tumor derived cells cultured as spheroid cultures and then treated with the indicated drugs for72h. Following 72 hours of treatment of the mammospheres, cells were dissociated with Acutase treatment. Following washing, cells were stained with CD24-PE & CD44-FITC antibody, washed and analyzed by FACS. The subpopulation of CD44+/CD24-low cells were gated and quantified. A significant effect was observed on CD44+/CD24low cells with salinomycin and salinomycin-NPs; not much effect was observed with Paclitaxel (see FIG. 18).

### Example 8

### Effects of Salinomycin-NPs on Tumor Growth Inhibition in an Animal Xenograft Mouse Model

The ability of salinomycin-containing nanoparticles to inhibit growth of H69 small cell lung carcinoma cells implanted in mice was examined.

Four to six-week-old Balb/c nu/nu mice were injected subcutaneously with 5X10⁶ H69 small cell lung carcinoma cells in the left flank. Mice with established H69 tumors (90-120 mm³) were randomized into groups of 6 mice each and treated i.p. (i) each day with vehicle control or (ii) once each week with 5 mg/kg salinomycin-nanoparticles for 3 weeks. Tumors were measured every other day with calipers, and tumor volumes were calculated using the formula (AXB²)/0.5, where A and B are the longest and shortest tumor diameters, respectively. Statistical analysis of tumor volumes was performed by one-way ANOVA and the Dunnett test using Origin 8.0 (Origin Lab).

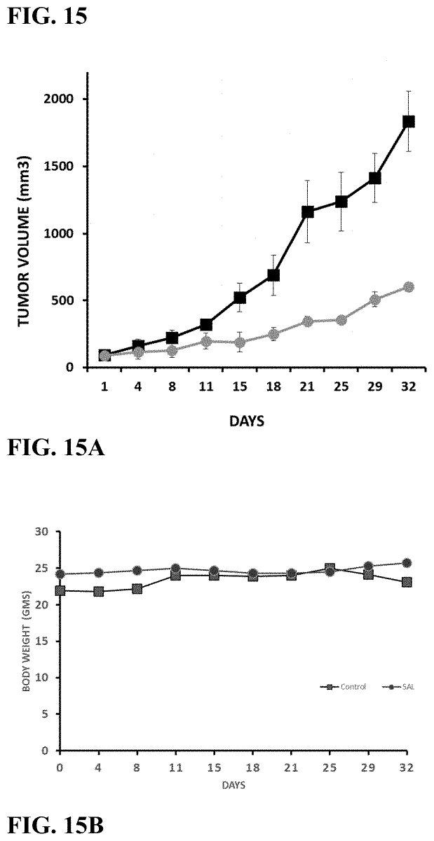

The results are shown in FIG. 15A. Shown is tumor volume (y-axis) over time (x-axis). Tumor volume in mice treated with vehicle control reached 2000 mm³. In contrast, tumor volume in mice treated with salinomycin-containing nanoparticles did not exceed 1000 mm³.

### Example 9

### Assessment of Body Weight in H69 Xenograft Mice Treated with Salinomycin-Containing Nanoparticles

The body weight of H69 xenograft mice treated with salinomycin-nanoparticles and control mice, as discussed in Example 6 above, was examined and compared to body weight of mice treated with vehicle over a period of 21 days.

The results are shown in FIG. 15B. Body weight remained stable or slightly increased in both groups during the length of the study. These results demonstrate that the salinomycin-containing nanoparticles do not adversely affect body weight.

### Example 10

### Comparative Toxicity in Wild-Type Mice of Varying Doses of Salinomycin and Salinomycin-Containing Nanoparticles

The effect of nanoparticles in mitigating the toxicity of salinomycin was examined in wild-type mice. Different doses of salinomycin alone (5, 7.5, 10, 12.5 and 15 mg/kg) or salinomycin-nanoparticles (SAL-NP) (5, 7.5, 10, 12.5 and 15 mg/kg) were injected into wild-type mice. Three mice were used in each group of salinomycin alone and salinomycin-NP groups, body weights, food and water uptake were measured every day for 22 days.

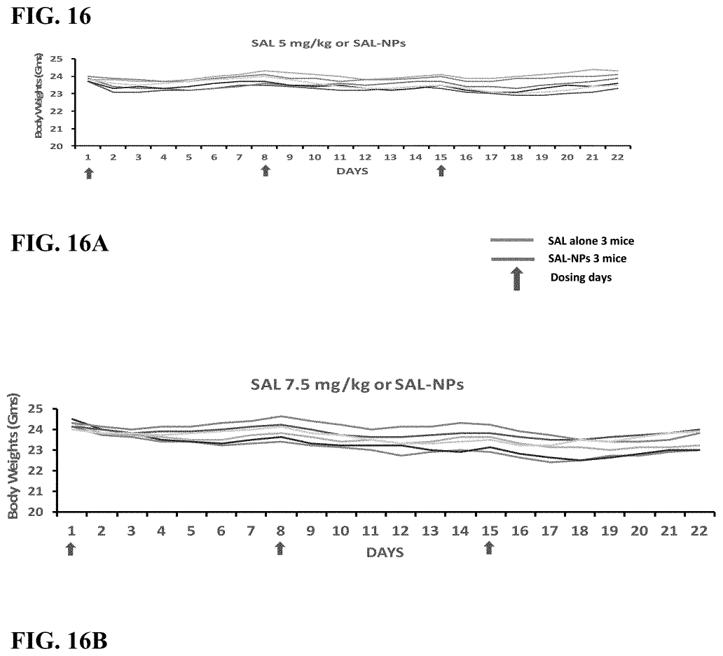

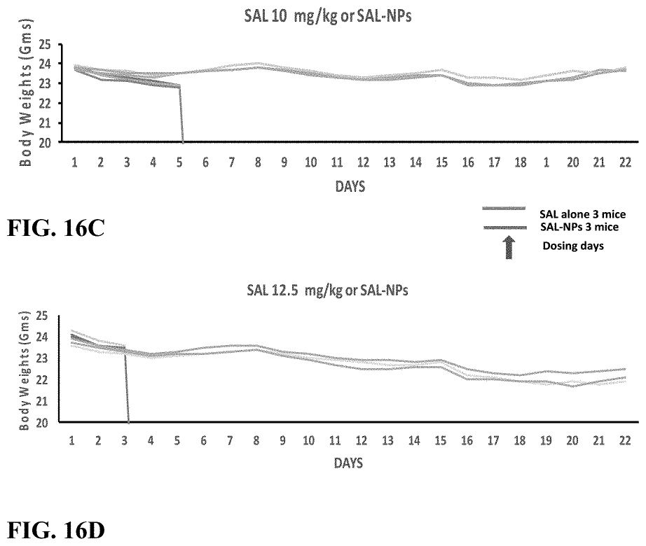

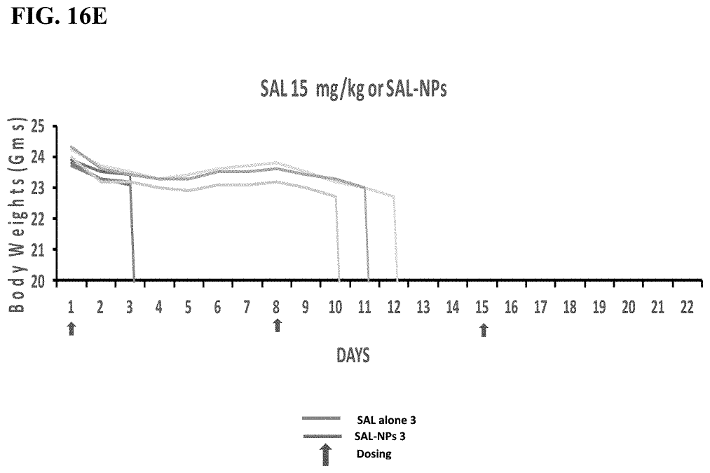

The results are shown in FIG. 16A-E. The results show body weight changes or lethality in mice treated with salinomycin alone and salinomycin-NPs. At the lowest doses, body weight was significantly higher at the end of the study in mice treated with salinomycin-nanoparticles relative to mice treated with salinomycin alone (FIG. 16A and 16B; 5 and 7.5 mg/kg doses, respectively).

At the next two higher doses tested, lethality was observed tested in mice treated with salinomycin alone; no mouse treated with salinomycin alone survived longer than five days (FIG. 16C; 10 mg/kg dose) or three days (FIG. 16D; 12.5 mg/kg dose). In contrast, mice treated with salinomycin-containing nanoparticles at these concentrations survived for the duration of the study with body weight essentially unchanged.

Lethality was observed in both groups at the highest concentration of salinomycin (FIG. 16E; 15 mg/kg). However, mice in the salinomycin-containing nanoparticle group survived until days 10-12 of the study, while all members of the group treated with salinomycin alone died after day 3.

These results demonstrate that nanoparticles mitigate the toxic effects of increasing doses of salinomycin in mice.

### Example 11

### Characterization of Salinomycin-Containing Nanoparticles

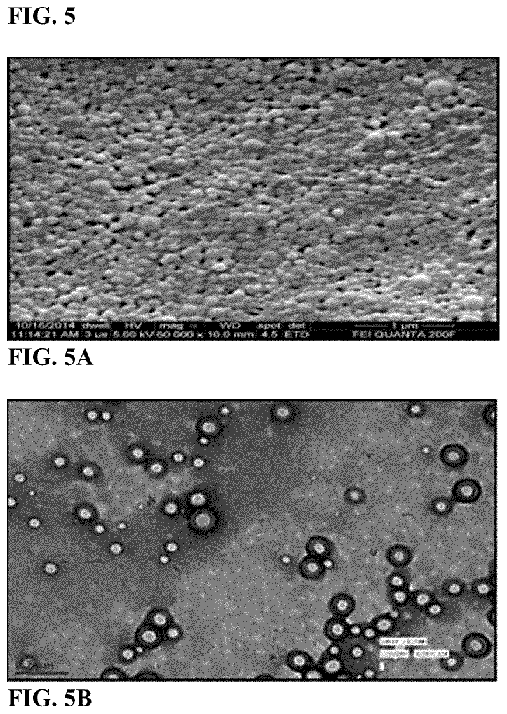

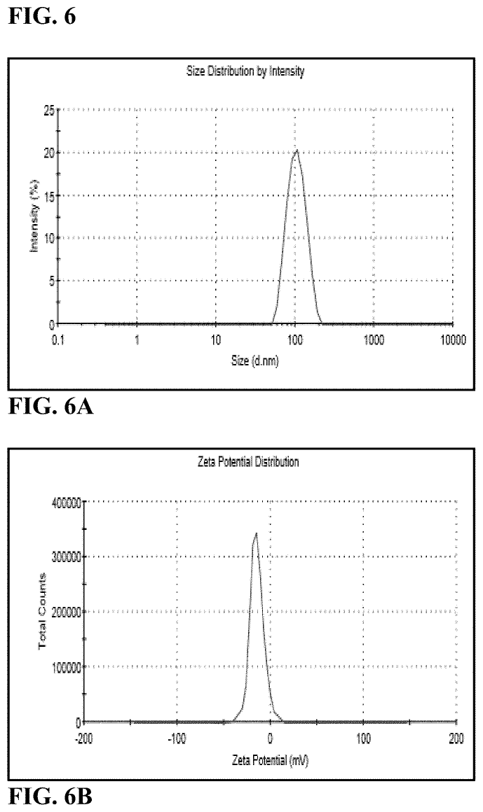

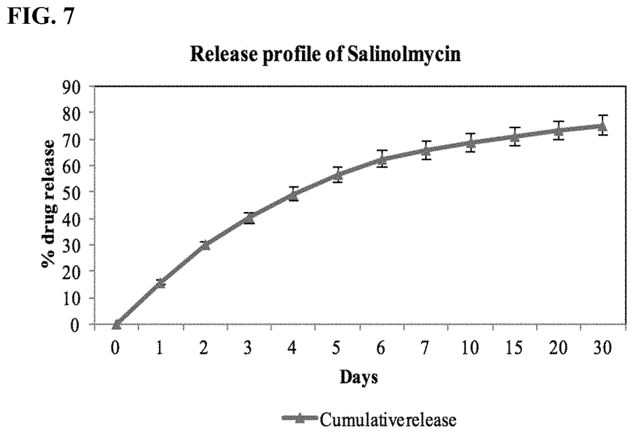

FIGS. 5A and 5B provide transmission electron micrographs providing the size and shape of the salinomycin-nanoparticles used in the Examples above. FIG. 7 is a graph showing the slow and sustained release of salinomycin from the nanoparticles over 30 days in an in vitro cell free buffer system.

FIG. 6A and FIG. 6B are graphs showing the size distribution and zeta potential distribution of salinomycin-nanoparticles. The physio-chemical characteristics of salinomycin-nanoparticles are detailed in Table 8 below and the gel permeation chromatography (GPC) of the co-polymers used is disclosed in Table 9 below.

TABLE 8
Physio-chemical Characteristics of SAL-NPs

| | Drug/polymer | | | |
|---|---|---|---|---|
| Samples | ratio | Size (nm) | Zeta P | PDI |
| PLA | — | 100.1 +/− 2.6 | −21.1 +/− 2.1 | 0.151 |
| PLA-PEG- | — | 102.1 +/− 2.1 | −13.9 +/− 1.3 | 0.072 |
| PPG-PEG | | | | |
| SAL-NPs | 1:10 | 108.5 +/− 3.6 | −16.14 +/− 1.8 | 0.059 |

TABLE 9
GPC of nanoparticles

GPC of Co-Polymers

| Samples | Mn (kD) | Mw (kD) | Mw/Mn |
|---|---|---|---|
| PLA^{72K} | 51,297 | 70,941 | 1.383 |
| PLA^{72K}-PEG-PPG-PEG | 67,557 | 83,293 | 1.242 |

1. A method of reducing proliferation, survival, migration, or colony formation ability of a rapidly proliferating cell in a subject in need thereof comprising contacting the cell with a therapeutically effective amount of a composition comprising
a) polymeric nanoparticles comprising a poly (lactic acid)-poly (ethylene glycol)-poly (propylene glycol)-poly (ethylene glycol) (PLA-PEG-PPG-PEG) tetra block copolymer, and;
b) salinomycin;
wherein the therapeutically effective amount is from about 0.025 mg/kg to about 5 mg/kg.

2. The method of claim 1, wherein the cell is a cancer cell or a cancer stem cell.

3. (canceled)

4. A method for treating cancer in a subject in need thereof comprising administering to the subject a therapeutically effective amount of a composition comprising
a) polymeric nanoparticles comprising a poly (lactic acid)-poly (ethylene glycol)-poly (propylene glycol)-poly (ethylene glycol) (PLA-PEG-PPG-PEG) tetra block copolymer, and;
b) salinomycin;
wherein the therapeutically effective amount is from about 0.025 mg/kg to about 5 mg/kg.

5. The method of claim 4, wherein the cancer is a non-metastatic or metastatic cancer selected from the group consisting of breast cancer, ovarian cancer, pancreatic cancer, leukemia, lymphoma, osteosarcoma, gastric cancer, prostate cancer, colon cancer, non-small cell lung cancer and small cell lung cancer, liver cancer, kidney cancer, head and neck cancer, and cervical cancer.

6. (canceled)

7. The method of claim 4, further comprising administering a second therapeutic agent, a targeted anti-cancer agent, or an additional anti-cancer therapy to the subject, wherein the subject is a human, and wherein the additional anti-cancer therapy is surgery, chemotherapy, radiation, hormone therapy, immunotherapy, or a combination thereof.

8. (canceled)

9. The method of claim 4, wherein the cancer is resistant or refractory to a chemotherapeutic agent.

10. (canceled)

11. A method of reducing proliferation, survival, migration, or colony formation ability of cancer stem cells in a subject in need thereof comprising administering to the subject a therapeutically effective amount of a composition comprising
a) polymeric nanoparticles comprising a poly (lactic acid)-poly (ethylene glycol)-poly (propylene glycol)-poly (ethylene glycol) (PLA-PEG-PPG-PEG) tetra block copolymer, and;
b) salinomycin;
wherein the therapeutically effective amount is from about 0.025 mg/kg to about 5 mg/kg.

12. The method of claim 1, wherein the therapeutically effective amount is from about 0.03 mg/kg to about 0.5 mg/kg, from about 0.5 mg/kg to about 0.8 mg/kg, or is between about 0.8 mg/kg to about 1.1 mg/kg.

13-14. (canceled)

15. The method of claim 1, wherein the composition is administered intravenously, intratumorally, or subcutaneously, wherein the composition is administered at least once per day, once every other day, once per week, twice per week, once per month, or twice per month, or wherein the composition is administered once per week or twice per week for a period of three weeks.

16-17. (canceled)

18. The method of claim 1, wherein the PLA-PEG-PPG-PEG tetra-block copolymer is formed from chemical conjugation of PEG-PPG-PEG tri-block copolymer with PLA.

19. The method of claim 1,
wherein the molecular weight of PLA is between about 10,000 and about 100,000 daltons, is between about 20,000 and 90,000 daltons, or is between about 30,000 and 80,000 daltons
wherein the molecular weight of PEG-PPG-PEG is between about 8,000 daltons and 18,000 daltons, or is between about 12,000 daltons and 17,000 daltons, or
wherein the molecular weight of PLA in the copolymer is between about 30,000 and 80,000 daltons and the molecular weight of PEG-PPG-PEG is 12,000 daltons and 17,000 daltons.

20-24. (canceled)

25. The method of claim 1, wherein the average diameter of the polymeric nanoparticles is between 80 and 120 nm, is between 90 and 110 nm, or is between 95 and 105 nm.

26-28. (canceled)

29. A pharmaceutical composition comprising
a) polymeric nanoparticles comprising a poly (lactic acid)-poly (ethylene glycol)-poly (propylene glycol)-poly (ethylene glycol) (PLA-PEG-PPG-PEG) tetra block copolymer;
b) salinomycin; and
c) a pharmaceutically acceptable carrier.

30. The pharmaceutical composition of claim 29, wherein the polymeric nanoparticle further comprises a targeting moiety attached to the outside of the polymeric nanoparticles.

31-45. (canceled)

46. The pharmaceutical composition for use according to claim 29, wherein the composition is formulated to be administered intravenously, intratumorally, or subcutaneously, optionally wherein the pharmaceutical composition comprises a dosage form from about 12.5 mg to about 500 mg.

47. The pharmaceutical composition for use according to claim 29, wherein the composition is formulated to be administered at least once per day, once every other day, once per week, twice per week, once per month, or twice per month, or is formulated to be administered once per week or twice per week for a period of three weeks.

48. (canceled)

49. The pharmaceutical composition for claim 29, wherein the PLA-PEG-PPG-PEG tetra-block copolymer is formed from chemical conjugation of PEG-PPG-PEG tri-block copolymer with PLA.

50. The pharmaceutical composition for use according to claim 29,
wherein the molecular weight of PLA is between about 10,000 and about 100,000 daltons, is between about 20,000 and 90,000 daltons, or is between about 30,000 and 80,000 daltons, or
wherein the molecular weight of PEG-PPG-PEG is between about 8,000 daltons and 18,000 daltons, or is between about 12,000 daltons and 17,000 daltons, or
wherein the molecular weight of PLA in the copolymer is between about 30,000 and 80,000 daltons and the molecular weight of PEG-PPG-PEG is 12,000 daltons and 17,000 daltons.

51-55. (canceled)

56. The pharmaceutical composition for use according to claim 29, wherein the average diameter of the polymeric nanoparticles is between 80 and 120 nm, is between 90 and 110 nm, or is between 95 and 105 nm.

57-58. (canceled)

59. The pharmaceutical composition for use according to claim 29, further comprising a second therapeutic agent or a targeted anti-cancer agent.
